(19) United States
(12) Patent Application Publication
(10) Pub. No.: US 2026/0076574 A1
(43) Pub. Date: Mar. 19, 2026

(54) APPARATUS AND METHODS TO ASSESS SUB-SURFACE TISSUE PROPERTIES

(71) Applicant: Board of Regents for Oklahoma Agriculture and Mechanical Colleges, Stillwater, OK (US)

(72) Inventor: Daqing Piao, Stillwater, OK (US)

(21) Appl. No.: 19/399,211

(22) Filed: Nov. 24, 2025

Related U.S. Application Data
(63) Continuation of application No. PCT/US2024/031946, filed on May 31, 2024.

(51) Int. Cl. A61B 5/00

(52) U.S. Cl. CPC A61B 5/0075; A61B 2562/0238; A61B 2562/0242; A61B 2562/185

(57) ABSTRACT

Absorption spectrophotometers are described herein. The absorption spectrophotometer comprises an illumination source and first and second spectrophotometers. The illumination source provides light to a point-of-illumination on a surface of a medium having a first layer and a second layer. The first spectrophotometer has an optical element and a first sensor. The optical element collects first reflected light passing through the first layer and emitted from the surface. The first sensor measures intensity of the first reflected light. The second spectrophotometer comprises a shroud, a center blocking module, and a second sensor. The center blocking module is supported within the shroud, forms a gap, and blocks first reflected light passing through the first layer emitted from the surface. The gap is sized and shaped to pass second reflected light passing through the deeper tissue and emitted from the surface. The second sensor measures intensity of the second reflected light.

### CROSS-REFERENCE TO RELATED APPLICATIONS

This application is a continuation of the international patent application identified by Serial No. PCT/US24/31946, filed on May 31, 2024, which claims priority to the provisional patent applications identified by U.S. Ser. No. 63/505,837, filed Jun. 2, 2023, and U.S. Ser. No. 63/626,382, filed Jan. 29, 2024, the entire contents of all of which are hereby expressly incorporated herein by reference.

### FEDERALLY SPONSORED RESEARCH AND DEVELOPMENT

This invention was made with government support under Grant No. 2022-67017-36538 awarded by the United States Department of Agriculture (USDA). The government has certain rights in the invention.

### SUMMARY OF THE INVENTION

In one aspect, the present disclosure includes an absorption spectrophotometer in combination with a medium having a surface, a first layer adjacent to the surface, and a second layer beyond the first layer, comprising: an illumination source operable to generate and provide a collimated beam of broadband light to a point-of-illumination (POI) on the surface of the medium; a first spectrophotometer having an optical element operable to collect a diffuse reflected beam of light passing solely through the first layer of the medium and emitted from the surface of the medium adjacent to the POI, and a first pixelated sensor to measure intensity of the diffuse reflected beam of light; and a second spectrophotometer comprising a shroud having an interior surface and a center blocking module having an exterior surface, the center blocking module coaxially supported within the shroud and forming a gap between the interior surface of the shroud and the exterior surface of the center blocking module, the center blocking module being sized, shaped, and constructed of an opaque material so as to block first diffuse reflected light passing solely through the first layer emitted from the surface of the medium adjacent to the POI, and the gap sized and shaped to pass second diffuse reflected light passing through the second layer and emitted from the surface of the medium at a predetermined range of distances from the POI, the second spectrophotometer also including a second pixelated sensor to measure intensity of the second diffuse reflected light emitted by the medium and passing through the gap between the interior surface of the shroud and the exterior surface of the center blocking module.

In another aspect, the present disclosure includes an absorption spectrophotometer, comprising: a first spectrophotometer having an optical element operable to collect a diffuse reflected beam of light within a first field-of-view (FOV), and a first pixelated sensor to receive and measure intensity of the diffuse reflected beam of light; and a second spectrophotometer comprising: a shroud positioned to receive diffuse reflected light within a second FOV overlapping the first FOV and surrounding the first FOV, the shroud having an interior surface; a center blocking module having an exterior surface, the center blocking module coaxially supported within the shroud and forming a gap between the interior surface of the shroud and the exterior surface of the center blocking module, the center blocking module being sized, shaped, and constructed of an opaque material so as to block first diffuse reflected light within a region of the second FOV overlapping the first FOV, the gap sized and shaped to pass second diffuse reflected light outside of the region overlapping the first FOV and within the second FOV; a second pixelated sensor operable to measure intensity of the second diffuse reflected light passing through the gap between the interior surface of the shroud and the exterior surface of the center blocking module; and an illumination source operable to generate and provide a collimated beam of broadband light to a POI on a surface of a medium within the first FOV and the second FOV.

### BRIEF DESCRIPTION OF THE DRAWINGS

The accompanying drawings, which are incorporated in and constitute a part of this specification, illustrate one or more implementations described herein and, together with the description, explain these implementations. The drawings are not intended to be drawn to scale, and certain features and certain views of the figures may be shown exaggerated, to scale or in schematic in the interest of clarity and conciseness. Not every component may be labeled in every drawing. Like reference numerals in the figures may represent and refer to the same or similar element or function. In the drawings:

FIG. 1 is a diagrammatic view of an exemplary implementation of an absorption spectrophotometer system constructed in accordance with the present disclosure;

FIG. 2 is a diagrammatic view of an exemplary implementation of an absorption spectrophotometer shown in FIG. 1;

FIG. 3 is a diagrammatic view of an exemplary implementation of a system device shown in FIG. 1;

FIG. 4 is a diagrammatic view of an exemplary implementation of a user device shown in FIG. 1;

FIG. 5 is a diagrammatic view of an exemplary implementation of a center-illuminated-center-blocked (CICB) configuration for non-contact diffuse reflectance spectroscopy (DRS) of a layer below a surface of a medium constructed in accordance with the present disclosure;

FIGS. 6A and 6B are perspective views of an exemplary implementation of a center blocking module constructed in accordance with the present disclosure;

FIG. 6C is a perspective view of another exemplary implementation of a center blocking module constructed in accordance with the present disclosure;

FIG. 6D is a plan view of a prior art annular aperture obstruction target;

FIG. 7 is a diagrammatic view of an exemplary implementation of a configuration for non-contact DRS of two layers at two different depths below the surface of the medium constructed in accordance with the present disclosure;

FIG. 8 is a diagrammatic view of another exemplary implementation of a configuration for non-contact DRS of two layers at two different depths below the surface of the medium constructed in accordance with the present disclosure;

FIG. 9 is a diagrammatic view of another exemplary implementation of a configuration for non-contact DRS of two layers at two different depths below the surface of the medium constructed in accordance with the present disclosure;

FIG. 10 is a diagrammatic view of another exemplary implementation of a configuration for non-contact DRS of two layers at two different depths below the surface of the medium constructed in accordance with the present disclosure; and

FIG. 11 is a diagrammatic view of a method of using the absorption spectrophotometer system shown in FIG. 1.

### DETAILED DESCRIPTION

The following detailed description refers to the accompanying drawings. The same reference numbers in different drawings may identify the same or similar elements.

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary-not exhaustive. Also, it is to be understood that the phraseology and terminology employed herein is for the purpose of description and should not be regarded as limiting.

Unless otherwise defined herein, scientific and technical terms used in connection with the presently disclosed inventive concept(s) shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification.

All patents, published patent applications, and non-patent publications mentioned in the specification are indicative of the level of skill of those skilled in the art to which this presently disclosed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

As utilized in accordance with the present disclosure, the following terms, unless otherwise indicated, shall be understood to have the following meanings:

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by anyone of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the inventive concept. This description should be read to include one or more and the singular also includes the plural unless it is obvious that it is meant otherwise.

Further, use of the term “plurality” is meant to convey “more than one” unless expressly stated to the contrary.

As used herein, qualifiers like “substantially,” “about,” “approximately,” and combinations and variations thereof, are intended to include not only the exact amount or value that they qualify, but also some slight deviations therefrom, which may be due to manufacturing tolerances, measurement error, wear and tear, stresses exerted on various parts, and combinations thereof, for example.

The use of the term “at least one” or “one or more” will be understood to include one as well as any quantity more than one. In addition, the use of the phrase “at least one of X, V, and Z” will be understood to include X alone, V alone, and Z alone, as well as any combination of X, V, and Z. The use of ordinal number terminology (i.e., “first”, “second”, “third”, “fourth”, etc.) is solely for the purpose of differentiating between two or more items and, unless explicitly stated otherwise, is not meant to imply any sequence or order or importance to one item over another or any order of addition.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, includes ranges of 1-20, 10-50, 50-100, 100-500, and 500-1,000, for example.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Circuitry, as used herein, may be analog and/or digital components, or one or more suitably programmed processors (e.g., microprocessors) and associated hardware and software, or hardwired logic. Also, “components” may perform one or more functions. The term “component,” may include hardware, such as a processor (e.g., microprocessor), an application-specific integrated circuit (ASIC), field-programmable gate array (FPGA), a combination of hardware and software, and/or the like. The term “processor” as used herein means a single processor or multiple processors working independently or together to collectively perform a task.

Software may include one or more computer readable instructions that when executed by one or more components cause the component to perform a specified function. It should be understood that the algorithms described herein (e.g., the mathematical model referred to in the attached document(s)) may be stored on one or more non-transitory computer readable medium. Exemplary non-transitory computer readable mediums may include random access memory, read only memory, flash memory, and/or the like. Such non-transitory computer readable mediums may be electrically based, optically based, and/or the like.

An illumination source, as described herein, includes a source of electromagnetic energy, such as an LED generating light, and one or more optical element (e.g., lens) for focusing or collimating the electromagnetic energy and optionally one or more device for directing the path of the electromagnetic energy. The device can be a conduit, a reflector, such as a mirror, one way mirror or the like, or a waveguide, such as an optical fiber. The electromagnetic energy can be in various parts of the electromagnetic spectrum, such as visible light, ultraviolet light or the like. The illumination source may be internal or external to the system described herein.

Functions described herein and in the attached disclosures may be performed by suitable component(s). For example, the first spectrophotometer and the second spectrophotometer may include one or more component for performing the functions described herein and in the attached disclosure(s) including analyzing data generated by the first spectrophotometer and the second spectrophotometer with the mathematical model as described in the attached document(s).

Turning now to the inventive concept(s), certain non-limiting embodiments thereof are described in the attached disclosures. While the attached disclosures describe the inventive concept(s) in conjunction with the specific drawings, experimentation, results, and language set forth hereinafter, it is evident that many alternatives, modifications, and variations will be apparent to those skilled in the art. Accordingly, it is intended to embrace all such alternatives, modifications, and variations that fall within the spirit and broad scope of the present disclosure.

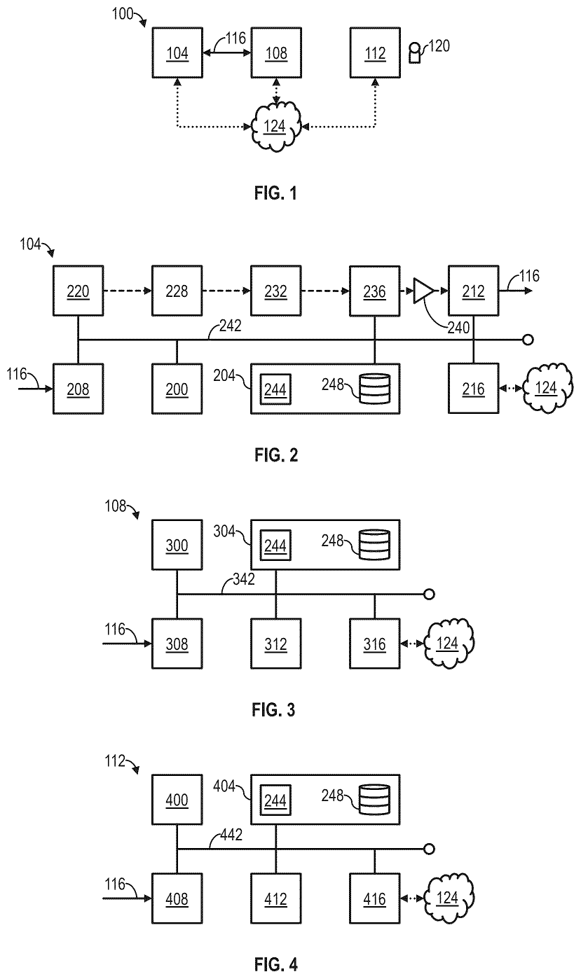

Referring now to the drawings and in particular to FIG. 1, shown therein is a diagrammatic view of an exemplary implementation of an absorption spectrophotometer system 100 (hereinafter, the “system 100”) constructed in accordance with the present disclosure. The system 100 may comprise one or more absorption spectrophotometer 104 (hereinafter, the “spectrophotometer 104”), one or more system device 108 (hereinafter, the “system device 108”), and one or more user device 112 (hereinafter, the “user device 112”).

The spectrophotometer 104 and the system device 108 may communicate data from the spectrophotometer 104 to the system device 108 and/or from the system device 108 to the spectrophotometer 104 via a data communication interface 116 configured to facilitate communication of information and/or data between the spectrophotometer 104 and the system device 108. In some implementations, the data communication interface 116 is a universal serial bus (USB) interface. However, in other implementations, the data communication interface 116 may be another form of data communication interface, such as serial advanced technology attachment (SATA), serial attached (SA) small computer system interface (SCSI) (SAS), peripheral component interconnect express (PCIe), Firewire, Ethernet, and/or the like.

A user 120 may interact with the system 100 using the user device 112, which may be used to request, such as from the system device 108, a graphical user interface (GUI) configured to accept input from the user 120 that may be transmitted to one or more of the system device 108 and the spectrophotometer 104. It is to be further understood that, as used herein, the term “user” is not limited to a human being, and may comprise a computer, a server, a website, a processor, a network interface, a human, a user terminal, a virtual computer, combinations thereof, and/or the like, for example.

The system device 108 may be connected to the user device 112 via a communication network 124. In implementations where the communication network 124 is the Internet, for example, the GUI of the system 100 may be delivered through a series of web pages or private internal web pages of a company or corporation, which may be written in hypertext markup language. However, it should be understood that the GUI of the system 100 may be another type of interface including, but not limited to, a Windows-based application, a tablet-based application, a mobile web interface, an application running on a mobile device, and/or the like.

The user device 112 may include, but is not limited to, implementation as a personal computer, a cellular telephone, a smart phone, a network-capable television set, a tablet, a laptop computer, a desktop computer, a network-capable handheld device, a server, a digital video recorder, a wearable network-capable device, and/or the like.

The number of components illustrated in FIG. 1 is provided for explanatory purposes. In practice, there may be additional components, fewer components, different components, or differently arranged components than are shown in FIG. 1. Furthermore, two or more of the components illustrated in FIG. 1 may be implemented within a single component, or a single component illustrated in FIG. 1 may be implemented as multiple, distributed components. Additionally, or alternatively, one or more of the components of the system 100 may perform one or more functions described as being performed by another one or more of the components of the system 100. Components of the system 100 may interconnect via wired connections, wireless connections, or a combination of wired and wireless connections.

Referring now to FIG. 2, shown therein is a diagrammatic view of an exemplary implementation of the spectrophotometer 104 shown in FIG. 1. The spectrophotometer 104 may comprise one or more processor 200 (hereinafter, the “instrument processor 200”), one or more non-transitory processor-readable medium 204 (hereinafter, the “instrument memory 204”), one or more input device 208 (hereinafter, the “instrument input device 208”), one or more output device 212 (hereinafter, the “instrument output device 212”), one or more communication device 216 (hereinafter, the “instrument communication device 216”), one or more illumination source 220 (hereinafter, the “illumination source 220”), a monochromator 228 (hereinafter, the “monochromator 228”), one or more medium 232 (hereinafter, the “medium 232”), one or more pixelated sensor 236 (hereinafter, the “sensor 236”), and one or more amplifier 240 (hereinafter, the “amplifier 240”).

Two or more of the instrument processor 200, the instrument memory 204, the instrument input device 208, the instrument output device 212, the instrument communication device 216, the illumination source 220, and the sensor 236 may be connected via an instrument path 242 (e.g., a data bus) that permits communication among the components of the spectrophotometer 104.

The instrument memory 204 may store one or more software application 244 (hereinafter, the “software application 244”) and/or one or more database 248 (hereinafter, the “database 248”). The software application 244 may be stored as processor-executable instructions that when executed by the instrument processor 200 cause the instrument processor 200 to actuate the illumination source 220 and receive sensor data from the sensor 236 to determine one or more property of the medium 232. The database 248 may be a relational database or a non-relational database. Examples of such databases comprise, DB2®, Microsoft® Access, Microsoft® SQL Server, Oracle®, MySQL, PostgreSQL, MongoDB, Apache Cassandra, and the like. It should be understood that these examples have been provided for the purposes of illustration only and should not be construed as limiting the presently disclosed inventive concepts. The database 248 may be centralized or distributed across multiple systems.

In some implementations, the spectrophotometer 104 may comprise one or more of the instrument processor 200 working together, or independently, to execute processor-executable instructions (e.g., the software application 244) stored in the instrument memory 204. Each element of the spectrophotometer 104 may be partially or completely network-based or cloud-based, and may or may not be located in a single physical location. The instrument processor 200 may be implemented as a single processor or multiple processors working together, or independently, to execute the software application 244 as described herein. It should be understood that in certain implementations using more than one of the instrument processor 200, each may be located remotely from one another, located in the same location, or comprising a unitary multi-core processor. The instrument processor 200 may be capable of reading and/or executing processor-executable code and/or capable of creating, manipulating, retrieving, altering, and/or storing data structures into the instrument memory 204. Exemplary implementations of the instrument processor 200 may include, but are not limited to, a digital signal processor (DSP), a central processing unit (CPU), a field programmable gate array (FPGA), a graphics processing unit (GPU), a microprocessor, a multi-core processor, combinations thereof, and/or the like, for example.

The instrument input device 208 may be capable of receiving information input from the user 120 and/or the instrument processor 200 and transmitting such information to other components of the spectrophotometer 104 and/or the system 100. The instrument input device 208 may include, but is not limited to, implementation as a keyboard, touchscreen, mouse, trackball, microphone, fingerprint reader, infrared port, slide-out keyboard, flip-out keyboard, cell phone, PDA, remote control, fax machine, wearable communication device, network interface, combinations thereof, and/or the like, for example.

The instrument output device 212 may be capable of outputting information in a form perceivable by the user 120 and/or the instrument processor 200, and/or the system device 108. For example, implementations of the instrument output device 212 may include, but are not limited to, a computer monitor, a screen, a touchscreen, a speaker, a website, a television set, a smart phone, a PDA, a cell phone, a fax machine, a printer, a laptop computer, a data communication port, a wireless or wired communication device, and combinations thereof, and the like, for example.

It is to be understood that in some exemplary implementations, the instrument input device 208 and the instrument output device 212 may be implemented as a single device, such as, for example, a touchscreen of a computer, a tablet, or a smartphone.

The instrument processor 200 may be capable of interfacing and/or communicating with the system device 108 and/or the user device 112 via the communication network 124 using the instrument communication device 216. For example, the instrument processor 200 may be capable of communicating via the communication network 124 by exchanging signals (e.g., analog, digital, optical, and/or the like) via one or more ports (e.g., physical or virtual ports) using a network protocol to interface and/or communicate with the system device 108 and/or the user device 112. In some implementations, the instrument processor 200 may be further capable of interfacing and/or communicating with the system device 108 and/or the user device 112 via the data communication interface 116. In such implementations, a particular one of the instrument input device 208 may be configured to receive information and/or data via the data communication interface 116, and a particular one of the instrument output device 212 may be configured to transmit information and/or data via the data communication interface 116.

The communication network 124 may permit bi-directional communication of information and/or data between the spectrophotometer 104, the system device 108, and/or the user device 112. The communication network 124 may interface with the spectrophotometer 104, the system device 108, and/or the user device 112 in a variety of ways. For example, in some implementations, the communication network 124 may interface by optical and/or electronic interfaces, and/or may use a plurality of network topographies and/or protocols including, but not limited to, Ethernet, TCP/IP, circuit switched path, combinations thereof, and/or the like. For example, in some implementations, the communication network 46 may be implemented as the World Wide Web (or Internet), a local area network (LAN), a wide area network (WAN), a metropolitan network, a 4G network, a 5G network, a satellite network, a radio network, an optical network, a cable network, a public switch telephone network, an Ethernet network, combinations thereof, and/or the like, for example. Additionally, the communication network 124 may use a variety of network protocols to permit bi-directional interface and/or communication of data and/or information between the spectrophotometer 104, the system device 108, and/or the user device 112.

Referring now to FIG. 3, shown therein is a diagrammatic view of an exemplary implementation of the system device 108 shown in FIG. 1. The system device 108 may comprise one or more processor 300 (hereinafter, the “system processor 300”), one or more non-transitory processor-readable medium 304 (hereinafter, the “system memory 304”), one or more input device 308 (hereinafter, the “system input device 308”), and one or more output device 312 (hereinafter, the “system output device 312”), and one or more communication device 316 (hereinafter, the “system communication device 316”). The system memory 304 may store the software application 244 and/or the database 248.

Two or more of the system processor 300, the system memory 304, the system input device 308, the system output device 312, and the system communication device 316 may be connected via a system path 342 (e.g., a data bus) that permits communication among the components of the system device 108. In some implementations, the system device 108 may comprise one or more of the system processor 300 working together, or independently, to execute processor-executable instructions (e.g., the software application 244) stored in the system memory 304. Each element of the system device 108 may be partially or completely network-based or cloud-based, and may or may not be located in a single physical location. The system processor 300 may be implemented as a single processor or multiple processors working together, or independently, to execute the software application 244 as described herein. It should be understood that in certain implementations using more than one of the system processor 300, each may be located remotely from one another, located in the same location, or comprising a unitary multi-core processor. The system processor 300 may be capable of reading and/or executing processor-executable code and/or capable of creating, manipulating, retrieving, altering, and/or storing data structures into the system memory 304. Exemplary implementations of the system processor 300 may include, but are not limited to, a digital signal processor (DSP), a central processing unit (CPU), a field programmable gate array (FPGA), a graphics processing unit (GPU), a microprocessor, a multi-core processor, combinations thereof, and/or the like, for example.

The system input device 308 may be capable of receiving information input from the user 120 and/or the system processor 300 and transmitting such information to other components of the system device 108 and/or the system 100. The system input device 308 may include, but is not limited to, implementation as a keyboard, touchscreen, mouse, trackball, microphone, fingerprint reader, infrared port, slide-out keyboard, flip-out keyboard, cell phone, PDA, remote control, fax machine, wearable communication device, network interface, combinations thereof, and/or the like, for example.

The system output device 312 may be capable of outputting information in a form perceivable by the user 120 and/or the system processor 300. For example, implementations of the system output device 312 may include, but are not limited to, a computer monitor, a screen, a touchscreen, a speaker, a website, a television set, a smart phone, a PDA, a cell phone, a fax machine, a printer, a laptop computer, combinations thereof, and the like, for example.

It is to be understood that in some exemplary implementations, the system input device 308 and the system output device 312 may be implemented as a single device, such as, for example, a touchscreen of a computer, a tablet, or a smartphone.

The system processor 300 may be capable of interfacing and/or communicating with the system device 108 and/or the user device 112 via the communication network 124 using the system communication device 316. For example, the system processor 300 may be capable of communicating via the communication network 124 by exchanging signals (e.g., analog, digital, optical, and/or the like) via one or more ports (e.g., physical or virtual ports) using a network protocol to interface and/or communicate with the spectrophotometer 104 and/or the user device 112. In some implementations, the system processor 300 may be further capable of interfacing and/or communicating with the spectrophotometer 104 and/or the user device 112 via the data communication interface 116. In such implementations, a particular one of the system input device 308 may be configured to receive information and/or data via the data communication interface 116, and a particular one of the system output device 312 may be configured to transmit information and/or data via the data communication interface 116.

Referring now to FIG. 4, shown therein is a diagrammatic view of an exemplary implementation of the user device 112 shown in FIG. 1. The user device 112 may comprise one or more processor 400 (hereinafter, the “user processor 400”), one or more non-transitory processor-readable medium 404 (hereinafter, the “user memory 404”), one or more input device 408 (hereinafter, the “user input device 408”), and one or more output device 412 (hereinafter, the “user output device 412”), and one or more communication device 416 (hereinafter, the “user communication device 416”). The user memory 404 may store the software application 244 and/or the database 248.

Two or more of the user processor 400, the user memory 404, the user input device 408, the user output device 412, and the user communication device 416 may be connected via a user path 442 (e.g., a data bus) that permits communication among the components of the user device 112. In some implementations, the user device 112 may comprise one or more of the user processor 400 working together, or independently, to execute processor-executable instructions (e.g., the software application 244) stored in the user memory 404. Each element of the user device 112 may be partially or completely network-based or cloud-based, and may or may not be located in a single physical location. The user processor 400 may be implemented as a single processor or multiple processors working together, or independently, to execute the software application 244 as described herein. It should be understood that in certain implementations using more than one of the user processor 400, each may be located remotely from one another, located in the same location, or comprising a unitary multi-core processor. The user processor 400 may be capable of reading and/or executing processor-executable code and/or capable of creating, manipulating, retrieving, altering, and/or storing data structures into the user memory 404. Exemplary implementations of the user processor 400 may include, but are not limited to, a digital signal processor (DSP), a central processing unit (CPU), a field programmable gate array (FPGA), a graphics processing unit (GPU), a microprocessor, a multi-core processor, combinations thereof, and/or the like, for example.

The user input device 408 may be capable of receiving information input from the user 120 and/or the user processor 400 and transmitting such information to other components of the user device 112 and/or the system 100. The user input device 408 may include, but is not limited to, implementation as a keyboard, touchscreen, mouse, trackball, microphone, fingerprint reader, infrared port, slide-out keyboard, flip-out keyboard, cell phone, PDA, remote control, fax machine, wearable communication device, network interface, combinations thereof, and/or the like, for example.

The user output device 412 may be capable of outputting information in a form perceivable by the user 120 and/or the user processor 400. For example, implementations of the user output device 412 may include, but are not limited to, a computer monitor, a screen, a touchscreen, a speaker, a website, a television set, a smart phone, a PDA, a cell phone, a fax machine, a printer, a laptop computer, combinations thereof, and the like, for example.

It is to be understood that in some exemplary implementations, the user input device 408 and the user output device 412 may be implemented as a single device, such as, for example, a touchscreen of a computer, a tablet, or a smartphone.

The user processor 400 may be capable of interfacing and/or communicating with the spectrophotometer 104 and/or the system device 108 via the communication network 124 using the user communication device 416. For example, the user processor 400 may be capable of communicating via the communication network 124 by exchanging signals (e.g., analog, digital, optical, and/or the like) via one or more ports (e.g., physical or virtual ports) using a network protocol to interface and/or communicate with the spectrophotometer 104 and/or the system device 108.

### 1.1. Introduction

Referring now to a first aspect of the present disclosure, the desire to assess the properties of a material below the layer that can be machine-visualized from the surface by non-contact (i.e., non-invasive and non-destructive) means is broadly applicable for quality control or monitoring purposes.

To assess the material properties by non-contact means, some form of energy (e.g., light, sound, electrical field, magnetic field, etc.) may be delivered to the material without physical contact of the applicator probe with the medium. The delivered form of energy after interacting with or being affected by the material may exit the medium, either at the same side of being delivered into the medium or at the opposing side (i.e., a distant part that is out of line-of-sight with respect to the location of delivery into the material) of the medium, for being measured. Comparing the properties (e.g., spectral intensity) of the energy leaving the material over the same property of the energy when delivered to the material may inform the properties of the material affecting the energy propagation in the material.

Optical spectroscopy utilizes light of multiple wavelengths or broad-band light to assess medium problems. Optical spectroscopy is implementable in either transmission mode or reflection mode. When measured in a reflection mode (i.e., measuring light exiting a medium at the same side into which the light has been introduced to the medium), the light exiting the medium to be collected for spectroscopy should not be mirror-reflected from the surface of the medium and must propagate within the medium before exiting for the light to be useful in assessing the properties of the medium below the surface of the medium. The resulting light exiting the medium, when compared to the light introduced to the medium, may be diffused.

Diffuse reflectance spectroscopy (DRS) refers to a method of utilizing light of multiple wavelengths or broad-band light to assess medium problems in a reflection mode. According to the principle of DRS, a multiple-wavelength or broad-wavelength light may be introduced to a medium, and the light emitting from the medium after propagating inside the medium may be compared against the light entering the medium by the system device 108, for example. The spectral intensity change may be used to inform the tissue properties. Placing the point-of-detection (POD) such that the POD overlaps with the point-of-illumination (POI) may cause the light path in the medium to be limited to a region near the surface. Conversely, placing the POD farther away from the POI may cause the light path in the medium to reach a deeper region; however, the light exiting the medium may be weaker due to taking a longer path and may experience greater attenuation in the medium. The general principle is that if the POI and the POD are separated by a distance of x, the light path will propagate through the medium up to a depth of ½ x. Therefore, providing a 1 mm distance between the POI and the POD would allow for light propagation up to 500 microns below the surface of the medium.

Accordingly, a broadband-spectrum light may be introduced into the medium at a small region of the size of 1 mm or less, for example, and then the light propagating through the medium may be measured by collecting at another small region of 1 mm or less, for example, that does not overlap with the POI. The path of the light that is injected into the medium at the POI and collected at the POD may interact with a region of the medium for the measurement to inform the property of the region. The greater the distance between the POD and the POI, the deeper the light path may interact. Therefore, to assess deeper regions, the light-path when propagating in the medium may reach that region of interest. Assessing deeper regions may require greater separation between the POI and the POD. More distant separation between the POI and the POD may cause the light at the POD to be weaker. Therefore, there is trade-off involved in moving the POD further from the POI to try and interrogate deeper regions, as the signal may become weaker, in some cases enough to fall below the noise of the instrument. Additionally, the light that has reached the deeper region must propagate through the shallower region. Therefore, the signal acquired at the longer POD that has interrogated the deeper region may be not only affected by the deeper region, but may be also affected by the shallower region. The effect of the shallower region, or the properties of the shallower region, may still need to be differentiated by the system device 108, for example.

To practically use DRS in non-contact mode to assess the properties of a medium below the region that can be grossly examined at the surface, the following constraints may be among those in play: (1) the light may be projected onto a surface region of the medium; (2) the light emitting from the medium may be coupled for detection off the surface of the medium; (3) the light emitting from the medium may contain components that have propagated into the region in which the property is to be assessed, and the light intensity may be within the dynamic range (i.e., the difference between the strongest and the weakest signal that can be detected) of instrument detection; and (4) a mathematical model may be used to connect how the tissue properties at the shallower and targeted region affect the light at the POD compared to the light at POI.

### 1.2. New Approaches to Non-Contact DRS

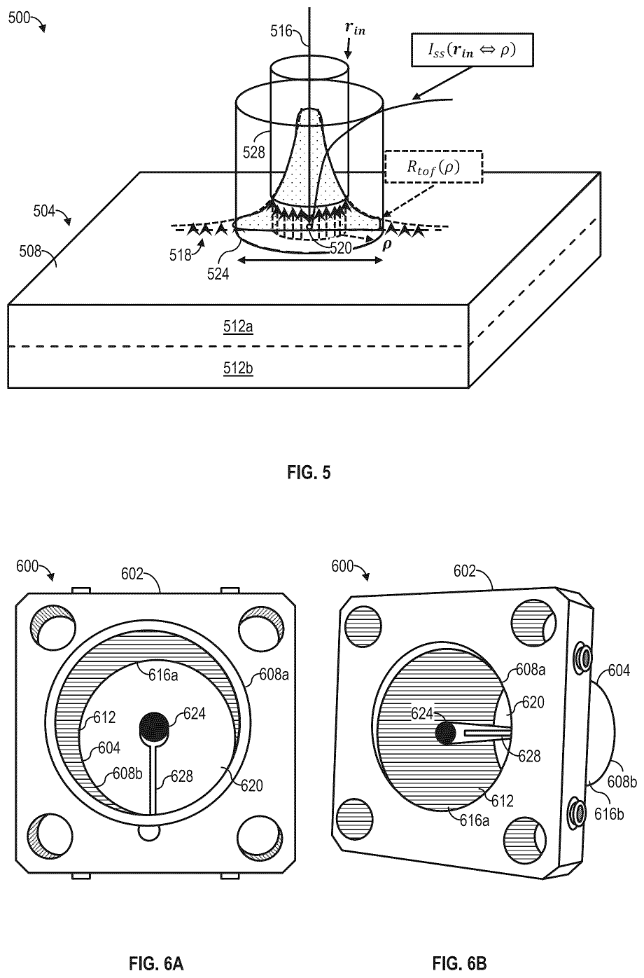

Referring now to FIG. 5, shown therein is an exemplary implementation of a configuration 500 of non-contact DRS referred to herein as center-illuminated-area-detection (CIAD). As shown in FIG. 5, the configuration 500 comprises a medium 504 having a surface 508, a first layer 512a adjacent to the surface 508, and a second layer 512b (the first layer 512a and the second layer 512b, collectively, the “layers 512”) beyond the first layer 512a. The first layer 512a is between the second layer 512b and the surface 508. In some implementations, the medium 504 is a biological tissue. In such implementations, the first layer 512a is shallow tissue, and the second layer 512b is deeper tissue. However, in other implementations, the medium 403 may be dye, paint, semiconductor materials, combinations thereof, and/or the like.

As described above, the spectrophotometer 104 may comprise the illumination source 220 operable to generate and provide a collimated beam of broadband incident light 516 (hereinafter, the “incident light 516”) to a point-of-illumination (POI) 520 on the surface 508 of the medium 504.

The configuration 500 shown in FIG. 5 differs from the conventional or the most common configuration of DRS in how diffusely reemitted light 518 (hereinafter, the “reemitted light 518”) (indicated by upward-facing arrows in FIG. 5) reemitting from the medium 504 is collected. When the incident light 516 is introduced into the medium 504, the reemitted light 518 may reemit everywhere from the surface 508 of the medium 504 within an area 524 centered around the POI 520. Instead of collecting the reemitted light 518 from a position away from the POI 520, CIAD may collect the reemitted light 518 from the entire area 524 centered around the POI 520, as is limited only by the optics for a given field-of-view (FOV). Because the reemitted light 518 is weaker the greater the distance away from the POI 520 the reemitted light 518 is collected, the totality of the reemitted light 518 collected over the area 524 may reach a saturating level that does not increase significantly with a further increase of the area 524.

CIAD may collect nearly all of the reemitted light 518 reemitted from the medium 504—thus increasing the total light intensity to make the signal-to-noise ratio (SNR) greater—and take the reemitted light 518 from the maximum area allowed by the optics, thus making the signal more sensitive to the absorption changes of the medium 504 to make it more desirable to sense the absorption properties of the medium 504; however, CIAD may have several limitations. For instance, CIAD may collect all of the reemitted light 518 in association with the injection of the incident light 516 at the POI 520, and because the collected signal may contain the reemitted light 518 that has passed long distances into the medium 504 (e.g., into the second layer 512b), the signal should theoretically respond to the properties of the second layer 512b; however, the signal may contain much stronger components of the reemitted light 518 that have passed short distances over a shallower region (e.g., the first layer 512a) close to the POI 520, and the signal may respond much more to the shallower or superficial properties of the medium 504 than to the deeper layer properties of medium 504.

To take advantage of the non-contact potential and the full-strength signal offered by the configuration 500 described herein to assess the deeper layer properties, the signal may be more sensitive to the deeper layer properties than to shallower properties. To make that happen, the reemitted light 518 having traveled through the first layer 512a may be removed from the signal being detected. To do that, a center portion 528 of the reemitted light 518 may be blocked from being detected, while not affecting the incident light 516. A hypothetical geometry of the projected configuration may be referred to herein as “center-illuminated-center-blocked (CICB) DRS” and may be configured for non-contact measurements of properties of the medium 504.

Should the center portion 528 of the reemitted light 518 be uncollected by the light detection, the strongest signal of the reemitted light 518 near the POI 520 that is most sensitive to the near-surface properties (i.e., properties of the first layer 512a) of the medium 504 may be removed, thus making the reemitted light 518 collected to be more sensitive to the deeper properties (i.e., properties of the second layer 512b) of the medium 504; however, the effect of the shallower medium properties may not be completely removed because the reemitted light 518 collected still passes through the first layer 512a below the area 524 where the reemitted light 518 is collected. By blocking the center portion 528 of the reemitted light 518 from being collected, it may be much more robust to assess the deeper layer properties of the medium 504.

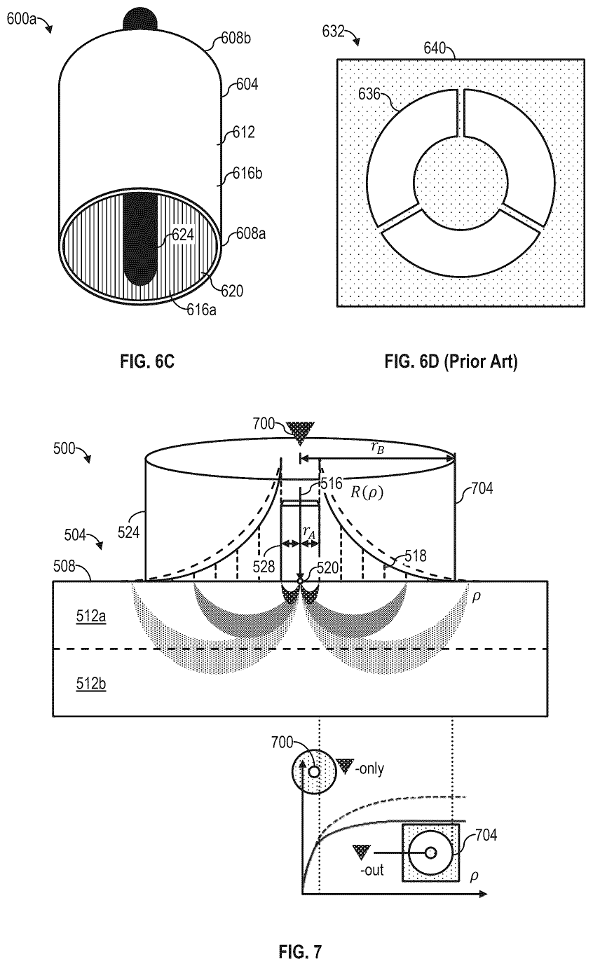

The center portion 528 of the reemitted light 518 may be incapable of being blocked without blocking the incident light 516. Therefore, the configuration 500 may be changed such that the reemitted light 518 is collected at an angle between 15 degrees and 60 degrees with respect to the axis of the incident light 516, such that the mechanism blocking the center portion 528 of the reemitted light 518 from entering the detection (i.e., a center blocking module 600 shown in FIGS. 6A-6C and discussed in more detail below) may not be in the way of the incident light 516 illuminated into the medium 504. Additionally, to make it possible to resolve the deeper layer properties (i.e., properties of the second layer 512b), the shallower layer properties (i.e., properties of the first layer 512a) may be assessed, which may be realized by collecting the reemitted light 518 in the center portion 528 that has been blocked from entering the CICB channel 704 (shown in FIG. 7). This may require two channels of light collection: one channel collecting light over a small area centered at and close to the POI 520 (i.e., the center portion 528) (i.e., the CICA channel 700 (shown in FIG. 7)), and the other channel collecting light over a large area centered at the POI 520 but rejecting the reemitted light 518 from the area 524 from which the CICA channel 700 collects light (i.e., the CICB channel 704).

### 1.3. How does the Center Blocking Module 600 Differ from Existing Arts for Blocking the Center Light Path?

Various implementations of the center blocking module 600 are shown in FIGS. 6A-6C. The center blocking module 600 may have a housing 602 (shown in FIGS. 6A and 6B) designed and built to fit into a standard 1″ cage optical system. However, in some implementations, the center blocking module 600 does not have the housing 602 (shown in FIG. 6C).

The center blocking module 600 may further comprise a tube 604 having a tube first end 608a, a tube second end 608b opposite the tube first end 608a, and a tube sidewall 612 extending between the tube first end 608a and the tube second end 608b, the tube sidewall 612 defining a shroud 614 and having a tube inner surface 616a and a tube outer surface 616b, the tube inner surface 616a defining a tube inner bore 620, and a rod 624 positioned within the tube inner bore 620 and attached to the tube inner surface 616a by one or more rod support 628 (hereinafter, the “rod support 628”).

In some implementations, the rod 624 has a first cross-sectional distance, and the shroud 614 has a second cross-sectional distance, wherein the first cross-sectional distance is in a range from 1/20 to ⅕ of the second cross-sectional distance.

The tube 604 may have a tube inner diameter in a range between 10 mm and 50 mm. In some implementations, the tube inner diameter is 1 inch (i.e., 25.4 mm), although the tube inner diameter can vary. The rod 624 may have a rod diameter in a range between 1 mm and 10 mm. In some implementations, the rod diameter is 3 mm.

The center blocking module 600 may be configured to block the center portion 528 of the reemitted light 518 from transmitting along the tube 604. While the center blocking module 600 is shown as being suspended or held in position by the rod support 628, it should be understood that the center blocking module 600 may be capable of being suspended or held in position without any physical connection with the tube 604.

The center blocking module 600 described herein may differ from prior art annular aperture obstruction targets-such as a prior art annular aperture obstruction target 632 shown in FIG. 6D-in aspects including but not limited to the following: (1) prior art annular aperture obstruction targets 632 are pin-hole type, and the diameter of the passing aperture 636 is 1 mm, while the center blocking module 600 described herein may have the tube inner diameter of the passing aperture that is 1 inch (i.e., 25.4 mm); (2) prior art annular aperture obstruction targets 632 are thin passing apertures 636 fabricated on transparent materials 640, while the center blocking module 600 described herein has the rod 624 of long aspect ratio (e.g., a length-to-width ratio between 0.5 and 50) mounted along the axis of the tube 604; and (3) prior art annular aperture obstruction targets 632 are used at the front of an imaging camera at the end of the optical path from the medium 504, or distal from the medium 504 from which the reemitted light 518 will be collected or imaged, while the center blocking module 600 described herein may be used at the beginning of the optical path from the medium 504, or proximal to the medium 504 from which the reemitted light 518 will be collected.

### 1.4. What does the CICB DRS Differ from Existing Arts of Dark-Field Microscopy?

Dark-field microscopy is an existing art. The CICB light differs than dark-field microscopy optical path in the following aspects: (1) spectroscopy versus microscopy; (2) dark-field microscopy blocks the center portion 528 of the incident light 516 while CICB DRS blocks the center portion of the reemitted light 118 emitting from the medium 504 from being collected, not the incident light 516 illuminating the medium 504; and (3) dark-field microscopy works in a transmission mode (i.e., the reemitted light 518 collected by the spectrophotometer 104 is at the opposite side of the light illuminating the medium 504). In some embodiments, the CICB DRS works in reflection mode (i.e., the reemitted light 518 collected by the spectrophotometer 104 is from the surface 508 that received the light illuminating the medium 504.

### 2.1. Methods

Referring now to a second aspect of the present disclosure by way of example, consumers discriminate against surface discoloration resulting in meat products being discounted or removed from the retail case. A recent study estimates surface discoloration costs the US beef industry approximately $3.73 billion annually. Interestingly, meat discoloration starts between the bright-red oxymyoglobin (OxyMb) layer and the interior deoxymyoglobin (DeoxyMb), where oxygen partial pressure is not enough to oxygenate all DeoxyMb molecules. More specifically, myoglobin is more prone to oxidation at a lower oxygen partial pressure.

After animal harvest, oxygen diffusion from the surface is the primary source of oxygen. Various factors-such as pH, fiber type, and mitochondrial activity-can influence oxygen diffusion into the interior. Diffusion of oxygen beneath the surface creates an oxygen gradient with greater oxygen at the surface and lower oxygen partial pressure in the interior of meat. The sub-surface oxygen partial pressure creates an ideal situation for metmyoglobin (MetMb) formation when MetMb reducing capacity decreases. Since interior discoloration may precede surface discoloration, measuring sub-surface myoglobin forms helps to characterize meat color changes. Nevertheless, limited techniques are currently available to study the interior discoloration of intact meat.

Various techniques have been used to evaluate the surface color of steaks during storage. The most common instrument for surface color evaluation is portable spectrophotometers. However, handheld spectrophotometers' light penetration is insufficient to quantify localized interior discoloration. The objective was to gain knowledge of in-situ sub-surface myoglobin forms of beef psoas major muscles during retail storage, by using an absorption spectrophotometer constructed in accordance with the present disclosure. The knowledge would help develop approaches towards process applicability.

### 2.2. Methods/Materials

The pHs of seven United States Department of Agriculture (USDA)-graded Select psoas major muscles (Institutional Meat Purchasing Specification No. 190A) were measured at three random locations across each tenderloin with a pH probe. Muscles were sliced into 1.91 cm thick steaks, and one steak from the anterior end was selected for analysis using needle-probe SfR spectroscopy and color measurements (the same steaks were used for needle-probe and color measurements). The remaining steaks were randomly assigned to analysis on day 0 or day 3 of the display, and used to measure MetMb reducing activity, oxygen penetration, and oxygen consumption. Steaks selected for retail display were packaged in white polystyrene polyvinyl chloride (PVC) overwrap foam trays. All steaks were placed in a coffin-style retail display case for five days at 2° Celsius (C). There was continuous LED lighting (12 watts (W), 48 inches, color temperature=3,500 Kelvin (K)) throughout the retail display.

### 2.3. Results

MetMb content increased during display from 1 mm to 5 mm depth. MetMb at a depth of 1 mm was greater compared to that of 2-5 mm depth. The sub-surface formation of MetMb aligns with changes in a* values during retail display and decrease in DeoxyMb content.

### 2.4. Prospects

Based on the results described above, it was determined that a closer source-detector separation (SDS) would make the detector more sensitive to the shallower myoglobin, and a farther SDS would make the detector more responsive to deeper myoglobin. Accordingly, the non-contact configuration of DRS using both closer and farther SDSes may help differentially resolve the deeper myoglobin.

### 3.1. Abstract

It is challenging to assess the spectral absorption of sub-surface medium using non-contact DRS. A demanding application of such is assessing myoglobin oxygenation at a depth of >1 mm to inform beef discoloration. Common to the broad-band DRS and especially challenging to non-contact DRS conducted in continuous-wave, probing the second layer 512b of the medium 504 introduces several geometry-dictated limitations in the measurements. For example, photons of longer pathlength are to be acquired to probe the second layer 512b of the medium 504. However, the information associated with the photons with paths specific to only the second layer 112b of the deep-probing path cannot be easily isolated in the measurement when additional information specific to only the first layer 512a is absent. Another challenge to instrumentation relates to the substantially different scales of the magnitudes of the shallower probing and deeper probing DRS signals when originating from the same illumination source 220 at a similar timeframe. We demonstrate a novel dual-channel non-contact DRS for the assessment of spectral absorption at a depth of below 1 mm. The dual-channel non-contact DRS combines a center-illuminated-center-acquired (CICA) geometry and a CICB geometry that are concentric with respect to the same POI 520. The CICA geometry enables probing the first layer 512a of less than 1 mm deep. And the CICB geometry acquires deep-probing photons while rejecting the short-path-shallow-probing-only components that would confound otherwise. The combination of CICA and CICB geometries allows assessing the below-surface spectral absorption. The principle is demonstrated by measurements from phantoms and tissues conforming to a two-layer geometry.

### 3.2. Introduction

Using two source-detector pairs differing in the SDS is a common approach in the widely applicable contact-based DRS for depth-responsive probing of the spectral absorption. The principle underlying this dual-SDS approach is that the average photon path corresponding to the greater SDS has propagated longer and also deeper into the medium 504 thereby may render information beyond what can be probed by the shorter SDS. Whereas the shorter SDS may be needed to resolve the spectral artifacts by the first layer 512a on the spectral properties of the second layer 512b of which the assessment generally requires a longer SDS.

In the case where the medium 504 is a homogenous medium, a longer photon path amplifies the spectral absorption variation over that of a shorter photon path, albeit by the same spatially uniform spectral modulation of the absorption over the photon passage. If the difference of the spectral diffuse reflectance from an unknown medium between acquired at two different SDSes deviates noticeably from a forward-prediction for the two SDSes based on a homogeneous medium, the homogeneity assumption is revised to indicate the presence of an anomaly. An anomaly to be sensed is often a localized mass of malignancy for biomedical applications but could also be a developing layer of absorption contrast, such as the formation of below-surface MetMb in packaged beef undergoing discoloration due to inadequate oxygen partial pressure and a doping heterogeneity in semiconductor materials causing bandgap disruption. For the former cases of localized mass of absorption anomaly, a checking of the spectral variation of the diffuse reflectance between acquired at two SDSes against the homogeneity-based forward predictions is considerably robust because of the well-established forward model of the simple geometry applying to DRS. For the latter cases of absorption difference occurring over the depth, the conventional strategy of using DRS acquired at two SDSes may become challenging in practice, particularly when requiring non-contact configurations. The spatially resolved, or equivalently radially resolved diffuse reflectance at the longer SDS, regardless of how its spectral variation might be in comparing to that of the shorter SDS, is likely orders of magnitude weaker than that of the shorter SDS should the two SDS differ significantly to probe much different depths. That may pose a problem of the photon budget over the dimension of the geometry of concern (i.e., the significant imbalance of the instrumental parameters needed to acquire at two much different SDSes). Whereas contact-based DRS of two SDSes is convenient by using fiber-optical probes, realizing two SDSes in non-contact DRS is uniquely challenging. Among the experimental non-contact DRS studies demonstrated to the present day, to the best of our knowledge, none has implemented two pairs of differing SDSes concurrently for the potential to assess whether the medium 504 probed may be heterogeneous over the depth. After all, non-contact DRS utilizing two different SDSes may be quite limited in how the two SDS can be realized.

One challenge of the non-contact DRS using two different SDSes may be to maintain two channels of optical projection to accommodate well-defined SDSes that may share the position of illumination or collection. This is beneficial to allow a relatively localized light path for the forward-model of radially resolved diffuse reflectance to be applicable to the measurement. To maintain well-defined SDSes in a non-contact setting, which, in some embodiments, requires spot-projection of both the illumination and collection light paths, also may result in a significant waste of the photon budget. This issue of photon budget is not exclusive to non-contact DRS.

However, in the contact-DRS, the issue of photon-budget is generally less concerned since it can be readily mitigated by using multiple fibers operating at the same SDS. For non-contact DRS, unfortunately, it may be difficult to find a balance between the benefit of spot-projection for better-defined SDS and the need to manage the dynamic range of the signals between the two channels providing differential responsiveness to the targeted depth.

Another challenge that is not unique to but more pronounced for non-contact DRS aiming to utilize two different SDSes is perhaps the difficulty to distinguish the effect of the spectral modulation of the first layer 512a on the spectral signal of the second layer 512b. A strategy of decoupling depends ultimately on the differential depth-responsivity of the two channels of different SDSes to the absorption variation at a depth. The spectral signal acquired with a longer SDS that has propagated into the second layer 512b must contain spectral modulation of the diffusely reflected light (i.e., the reemitted light 518) by the first layer 512a. A spectral contrast of the optical properties at a depth may affect the diffuse reflectance acquired over both the shorter and longer SDSes but must have different extents and possibly vary in opposite directions upon the same change of the properties of the medium 504. If only comparing the radially resolved diffuse reflectance corresponding to the shorter and longer SDSes, the response by the shorter SDS that interrogates the first layer 512a is inherently “embedded” in the response by the longer SDS. The responses of the two spectral signals acquired at the two SDSes to the same spectral contrast (i.e., both absorption and scattering) of the second layer 512b may differ in magnitude but will likely follow the same direction of variation (i.e., either increasing or decreasing versus the spectral change). This issue is also not exclusive to non-contact DRS but is more problematic in non-contact DRS because of the practical constraints on how the incident light 516 can be delivered unto and the reemitted light 518 collected from the medium 504.

The photon budget issue may be eased by collecting the diffuse reflectance over the area 524 of light remission centered on the POI 520 compared to taking a spot-localized diffuse reflectance. With such configuration, the total diffuse reflectance collected over an area of a greater radial dimension will always be greater than that collected over an area of smaller radial dimension and will also contain the reemitted light 118 that has propagated deeper than the other. Therefore, a spectral variation of the absorption at a depth will result in similar pattern of change of the two sets of the total diffuse reflectance at two radial dimensions. This may reduce the differential sensitivity of the two sets of total diffuse reflectance of different radial dimensions comparing to what could be obtained with two well-defined SDSes, due to the averaging of the radially resolved diffuse reflectance. But what if comparing the entirety of the diffuse reflectance near the POI 520 that has interrogated primarily the first layer 512a and the entirety of diffuse reflectance excluding those of the former to have sampled the second layer 512b containing a layer of absorption contrast? If such separation of the two values of diffuse reflectance can be done, will the central-excluded total diffuse reflectance respond differently to the spectral contrast between the first layer 512a and the second layer 512b than the central-included total diffuse reflectance?

Described herein is a novel configuration of non-contact DRS for the potential of probing at least two different depths, by utilizing two channels of area light collection over complementary regions associated with the same POI 520. This configuration sets one channel of non-contact DRS to collect the reemitted light 518 over the center portion 528 containing the POI 520 and another channel of non-contact DRS to collect the reemitted light 518 over the area 524 that is much greater in the outer dimension than the center portion 528 of the former channel and excludes the center portion 528 of the former channel. Such configuration makes the areas of light collection between the two channels complementary to each other, which makes the two channels respond more differentially to a deeper spectral contrast than would be rendered by two simple DRS channels differing in the SDSes only.

### 3.3. Principle of Operation

### 3.3.1. Geometry of Concern

The principle of the proposed configuration for utilizing two area-collection channels with non-contact DRS is illustrated in FIG. 6, concerning the diffuse light remission (i.e., the reemitted light 518) from a semi-infinite medium 504 that is under an idealized point illumination (i.e., the incident light 516). The radially resolved diffuse reflectance may decrease versus the radial distance, whereas the total diffuse reflectance—or the diffuse reflectance integrated over the area centered around the POI 520—may increase versus the radius of the area 524 until reaching a saturation. The channel A or the CICA channel 700 (hereinafter, the “CICA channel 700”) may represent the collection of the reemitted light 518 over the area 524 having a radius of r_{A} containing the POI 520. The channel B or the CICB channel 704 (hereinafter, the “CICB channel 704”) may represent the collection of the reemitted light 518 over the area 524 having an inner radius of r_{A} and an outer radius of r_{B}. The area of collection of the CICB channel 704 therefore may be complementary to that of the CICA channel 700 over the gross area that has an outer radius of r_{B}.

### 4.3.2. Model of the Total Diffuse Reflectance Associated with the CICA Channel 700 and the CICB Channel 704

A semi-infinite homogeneous medium 504—as is illustrated in FIG. 7—may be specified by a set of diffusivity-relevant parameters of

$$[ {μ}_{a} , {μ}_{s}^{′} ] ,$$

where μₐ is an absorption coefficient and

$${μ}_{s}^{′}$$

is a reduced scattering coefficient. The radially resolved diffuse reflectance evaluated at a distance of ρ from the POI 520 is denoted R(ρ). Accordingly, the total diffuse reflectance assessed over the area centered on the POI 520 and having a radius of r_{A} (i.e., the CICA channel 700) is:

$$I ( 0 ⇔ {r}_{A} ) = 2 π {\int }_{0}^{{r}_{A}} R ( {ρ}^{′} ) \cdot {ρ}^{′} \cdot d {ρ}^{′} \qquad ( 1 )$$

The I(0⇔r_{A}) in Equation (1) is also referred to as I_{A} as it is the total diffuse reflectance acquired by the CICA channel 700. The counterpart of Equation (1) by replacing r_{A} with r_{B} is then I(0⇔r_{B}). And the amount of I(0⇔r_{B}) that surpasses I(0⇔r_{A}) is the total diffuse reflectance that can be collected over the area of a radius of r_{B} after excluding the central area of a radius of r_{A} (i.e., the CICB channel 704). The total diffuse reflectance corresponding to the CICB channel 704 is I_{B} and is given by the following:

$${I}_{B} = I ( {r}_{A} ⇔ {r}_{B} ) = I ( 0 ⇔ {r}_{B} ) - I ( 0 ⇔ {r}_{A} ) \qquad ( 2 )$$

We consider the medium 504 as having two layers, of which the second layer 512b is numbered 0 and the first layer 512a is numbered 1. The set of the absorption and reduced scattering properties of the layers 512 are specified as

$${[ {μ}_{a} , {μ}_{s}^{′} ]}^{0 , 1} ,$$

wherein the superscripts mark the layer 512. Then for otherwise the same geometry, the set of diffuse reflectance defined by Equations (1) and (2) that correspond to a homogeneous medium 504 of the properties of either layer 0 or layer 1 are denoted as

$${I}_{A , B}^{0 , 1} ,$$

wherein the subscript differentiates the CICA channel 700 or the CICB channel 704 and the superscript distinguishes whether the property of the medium 504 is of

$${[ {μ}_{a} , {μ}_{s}^{′} ]}^{0} {or [ {μ}_{a} , {μ}_{s}^{′} ]}^{1} .$$

The total diffuse reflectance of the CICA channel 700 at a specific radial dimension will be bounded between

$${I}_{A}^{0} and {I}_{A}^{1}$$

at the same radial dimension. Similarly, the resulted total diffuse reflectance of the CICB channel 704 at a specific radial geometry will be bounded between

$${I}_{B}^{0} and {I}_{B}^{1}$$

at the same radial dimension. The total diffuse reflectance of the medium 504 can then be modeled as the following:

$${I}_{A , B}^{2} = {I}_{A , B}^{0} + [ {I}_{A , B}^{1} - {I}_{A , B}^{0} ] \cdot {Δ}_{layer} \qquad ( 3 )$$

$$where$$

$${Δ}_{layer} = [ 1 - ( 1 - {Δ}_{a} ) \cdot ( 1 - {Δ}_{s}^{′} ) ] \cdot {Δ}_{δ} \qquad ( 4 )$$

acts to weight how close the total diffuse reflectance from the medium 504 is to that of the second layer 512b or the first layer 512a, based on the contrast of the layers 512 and the thickness of the first layer 512a. In Equation (3), the Δₐ and

$${Δ}_{s}^{′}$$

are respectively an absorption-contrast factor and a scattering-contrast factor, and Δ_{δ} is a depth-weighting factor, defined respectively as the following:

$${Δ}_{a} = {( \frac{{μ}_{a}^{0} - {μ}_{a}^{1}}{{μ}_{a}^{0} + {μ}_{a}^{1}} )}^{2} \qquad ( 5 )$$

$${Δ}_{s}^{′} = {( \frac{{μ}_{s}^{′0} - {μ}_{s}^{′1}}{{μ}_{s}^{′0} + {μ}_{s}^{′1}} )}^{2} \qquad ( 6 )$$

$${Δ}_{δ} = {( \frac{δ}{10} )}^{γ} , γ is a user - definable number in [ 0 , 1 ] \qquad ( 7 )$$

It is straightforward to appreciate that Equation (3) will relax to

$${I}_{A , B}^{0}$$

when the optical contrast between the layers 512 vanishes or the first layer 512a disappears. And Equation (3) will approach

$${I}_{A , B}^{1}$$

when the optical contrast between the layers 512 is overwhelmed by the first layer 512a and the first layer 512a extends extensively into the second layer 512b. The form of

$${I}_{A , B}^{0 , 1}$$

takes the algebraic model presented in that is also modified slightly by multiplying with an empirical factor in the shape of [1−exp(−r_{A}/r₀)], where r₀ is a reference number chosen empirically, to suppress the over-estimation of the simple algebraic model at the sub-diffusive dimension.

### 3.3. Methods and Materials

### 3.3.1. Monte Carlo (MC) Simulation

MC simulations were conducted on a two-layer geometry with the first layer 512a having a fixed thickness of 1 mm. The MC simulations were used to examine how the CICA channel 700 and the CICB channel 704 respond to a differential contrast between the layers 512 of the medium 504, and how well the analytical model-approach specified above applies to the CICA channel 700 and the CICB channel 704. The MC solver produced the radially resolved diffuse reflectance from an air-bounding turbid medium of only the Heyney-Greenstein (HG) phase function specified at a scattering anisotropy of 0.9 due to the availability of the software platform. A user-editable input file was modified to customize the medium 504 geometry, medium 504 optical properties, and positions of evenly spaced evaluation points on the surface 508 of the medium 504. The total thickness of the medium 504 was set as 10 cm, and the refractive indices of both of the layers 512 were set as 1.40. The radially resolved diffuse reflectance was assessed at a radial interval of 1 μm over a total radial distance of 15 mm. A total of 100,000 photons were launched for each run or each combination of the medium 504. All photons arriving at a modeled surface point were counted toward the radially resolved diffuse reflectance, which was then assembled over each circular strip according to the differential area of the strip followed by summation over all circular strips to arrive at the total photon remission over the area of collection.

The baseline properties of each of the layers 512 were specified as μₐ=0.01 mm⁻¹ and

$${μ}_{s}^{′} = 1. {mm}^{- 1} .$$

The MC simulations were conducted at the following configurations of the properties of the first layer 512a and the second layer 512b: (1) the second layer 512b was at the baseline

$$( {μ}_{a} = 0.01 {mm}^{- 1} and {μ}_{s}^{′} = 1 {mm}^{- 1} )$$

and the first layer 512a had the absorption coefficient varying incrementally (i.e., 10 equally spaced values per decade) over four orders of magnitude, covering μₐ=[0.001, 0.002, . . . , 0.01, 0.02, . . . , 0.1, 0.2, . . . , 1, 2, . . . , 10] while

$${μ}_{s}^{′}$$

was at 0.1, 1, or 10 mm⁻¹, respectively; and (2) the second layer 512b was set at the baseline and the first layer 512a had the reduced scattering coefficient varying incrementally (i.e., 10 equally spaced valued per decade) over two orders of magnitude covering

$${μ}_{s}^{′} = [ 0.1 , 0.2 , ... , 1 , 2 , ... 10 ]$$

while μₐ was at 0.001, 0.01, 0.1, or 1 mm⁻¹, respectively. The r_{A} and r_{B} were set as 1.5 mm and 12.5 mm respectively, based on the experimental configuration.

### 3.3.2. Experimental Configuration

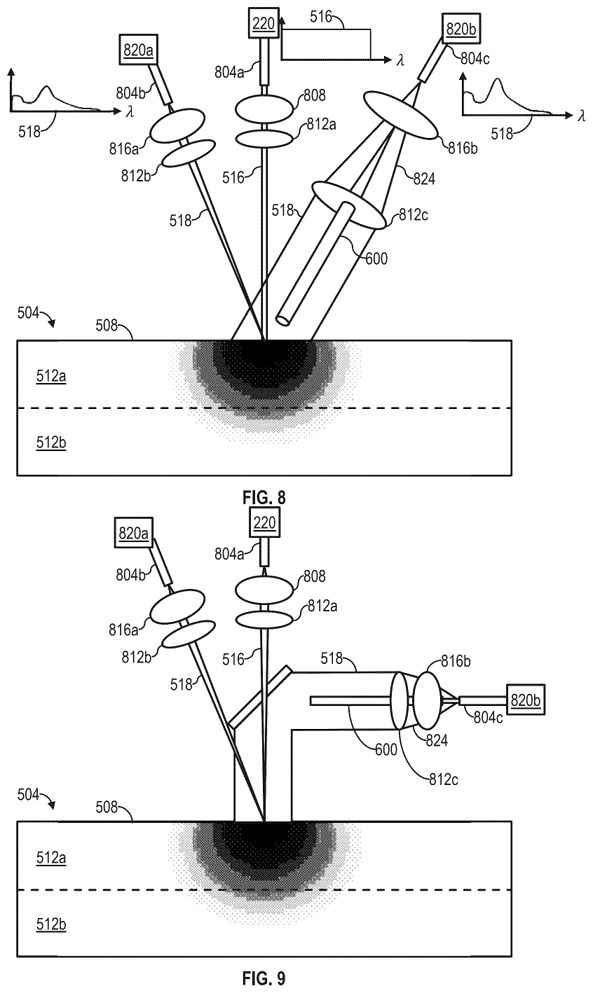

The experimental setup of the non-contact DRS that has configured the two channels of acquisition as conceptualized in FIG. 7 is shown in FIG. 8. The incident light 516 from a laser-driven illumination source 220 may be coupled by a first fiber 804a (e.g., a 200 μm fiber) to be projected normally onto the medium 504 via one or more first lens (e.g., a collimating lens 808 and a first relay lens 812a (e.g., focal length=200 mm) shown in FIG. 8). The reemitted light 518 may be acquired by two channels set at the opposite sides of the illuminating path and differing in the size of the area of collection. The CICA channel 700 may be aligned at an angle of approximately 22.5 degrees with respect to the optical axis of the illumination path. The CICB channel 704 may be aligned at an angle of approximately 30 degrees with respect to the optical axis of the illumination path. The CICA channel 700, which may comprise one or more second lens (e.g., a second relay lens 812b (e.g., focal length=100 mm) and a first focusing lens 816a shown in FIG. 8), may collect lights from an area of approximately 3 mm in diameter centered at the POI 520. The CICB channel 704, which may comprise one or more third lens (e.g., a third relay lens 812c (focal length=100 mm) and a second focusing lens 816b shown in FIG. 8), may collect lights from an area of approximately 25 mm in diameter centered at the POI 520. The centers of the two channels (i.e., the CICA channel 700 and the CICB channel 704) may be aligned to overlap with the POI 520 at the surface 508 of the medium 504. The CICB channel 704 may implement a center blocking module 600 attached to a tube (e.g., a 1″ diameter tube) at two longitudinal positions to align the center blocking module 600 to make the detection optics blind to the central region of, for example, 3 mm in diameter. The CICA channel 700 may be coupled via a second fiber 804b (e.g., a 400 μm fiber) to a first compact spectrophotometer 820a (spectral response=200-1000 nm). The CICB channel 704 may be coupled via a third fiber 804c to a second compact spectrophotometer 820b (custom spectral response=530-1000 nm) (the first compact spectrophotometer 820a and the second compact spectrophotometer 820b, collectively, the “spectrophotometers 820”). Both of the spectrophotometers 820 may be controlled via their respective user interfaces. Exposure times of the spectrophotometers 820 may range between 30 to 1000 ms, depending upon the medium. When prompted, the spectral profiles over a 10 nm range centered at 620 nm from both of the spectrophotometers 820 may be averaged to obtain one value of the total diffuse reflectance of the corresponding channel, at a specific condition of the single or substrate medium of which the scattering or absorption was varied. The spectrophotometers 820 may be constructed and used in a similar manner as the spectrophotometer 104.

The performance of the two channels of non-contact DRS were assessed with mediums (media) 504 of various forms but share the same first layer 512a, which was a 1/16 inch (˜1.6 mm) thick sliced cured ham. The use of the sliced cured ham as the first layer 512a rendered a relatively well-defined two-layer geometry with the second layer 512b controllable and more relevant to the targeted application. The spectral absorption of the 1/16 inch sliced cured ham was estimated by comparing the reduction of the spectral transmission after adding one layer of the sliced cured ham over a substrate in a custom-developed transmission spectrophotometry device. A total of 20 repeated measurements using samples randomly taken from the same package were averaged to arrive at the spectral absorption

$${μ}_{a}^{1} ( λ )$$

of the 1/16 inch thick cured ham. The estimation of the spectral absorption also considered the spatial angle or the numerical aperture of the fiber-optics in collecting the reemitted light 518 that transmitted through the slab of the sliced cured ham being a turbid medium. The reduced scattering spectrum of the sliced cured ham was estimated as

$${μ}_{s}^{′1} ( λ ) = {( λ / 1000 )}^{- 2.5} {mm}^{- 1} ,$$

where λ is the wavelength by nanometers, over a spectral range of [530, 900] nm within which the two the CICA channel 700 and the CICB channel 704 rendered useful information.

A randomly chosen slice of 1/16 inch thick cured ham was placed as the first layer 512a of the medium 504. Attached to the sliced cured ham was a substrate of a transparent food-packaging film for optical contact with the second layer 512b of the medium 504. The second layer 512b of the medium 504 was configured by one of the following approaches.

In a first approach, three solid phantoms having near-identical scattering and the absorption scaled at approximately 1:2:4 were placed as the second layer 512b in hard-contact with the first layer 512a.

In a second approach, an aqueous medium of increasing scattering was made by dissolving TiO₂ powders in 1000 mL water housed in a custom-made container having a second layer 512b of 1/16 inch thickness. The water container was made to have the surface level of the water in soft contact with the second layer 512b of the cured ham via the film. The container with water was placed on top of a magnetic stirrer to agitate the aqueous medium using a 1.5″ stirrer at a speed of 180 revolutions per minute (RPM). TiO₂ powder was added to the solution to reach the following cumulative weights: 0.01 g to 0.1 g at an increment of 0.01 g, 0.1 g to 1 g at an increment of 0.1 g, 1 g to 10 g at an increment of 1 g, and 10 g to 80 g at an increment of 10 g. The reduced scattering coefficient of the aqueous medium containing TiO₂ at 620 nm was estimated based on

$${μ}_{s} ( λ ) = {μ}_{s}^{′} ( λ ) = 0.39 {λ}^{- 1.4} {mm}^{- 1} per mg / ml of {TiO}_{2} ,$$

(wavelength in the unit of μm). At 620 nm, this gives

$${μ}_{s}^{′} = 0.762 {mm}^{- 1} per mg / ml of {TiO}_{2} .$$

In a third approach, an aqueous medium of increasing absorption was made by dissolving Indian ink to the last condition of the turbid medium of the second approach described above. The ink was injected with a 1 cc insulin syringe to reach the following cumulative volume in the host turbid medium: 0.01 to 0.1 mL at a step of 0.01 mL using a dropping technique, 0.1 mL to 1.0 mL at a step of 0.1 mL, and 1 mL to 5 mL at a step of 1 mL. The baseline μₐ of the TiO₂ solution at 620 nm was set as 0.002 mm⁻¹. The absorption coefficient of the ink was calculated. The upper surfaces of the samples were leveled manually to align against a pre-set position.

### 3.4. Results

3.4.1. MC and Model-Prediction of the Responses of the CICA Channel 700 and the CICB Channel 704 to the Differential Contrasts of the Layers 512 with a 1-Mm Thick First Layer 512a

The following patterns of the diffuse reflectance corresponding to the CICA channel 700 and the CICB channel 704 when responding to the change of the absorption of the first layer 512a can be observed. The MC simulation reveals that the diffuse reflectance of both the CICA channel 700 and the CICB channel 704 may reduce as the absorption of the first layer 512a increases, and the increase of the first layer 512a scattering may increase the intensity of the CICA channel 700 but decrease the intensity of the CICB channel 704. As the second layer 512b remained at the baseline, the change of the absorption of the first layer 512a may cause a change of the absorption contrast of the first layer 512a versus the second layer 512b. And at a given scattering of the first layer 512a, an increase of the absorption of the first layer 512a may reduce the amount of light that can propagate in the medium 504, regardless of the layer 512, and eventually reemit from the surface 508. Therefore, the diffuse reflectance signals of both the CICA channel 700 and the CICB channel 704 may reduce as the absorption of the first layer 512a decreases, whereas the amount of reduction may differ between the CICA channel 700 and the CICB channel 704 because the difference of the light paths between those reemitting over the region of the CICB channel 704 and that over the CICA channel 700.

The model-predictions of the CICA channel 700 and the CICB channel 704 as a function of the first layer 512a absorption at the baseline reduced scattering of

$${μ}_{s}^{′} = 1. {mm}^{- 1}$$

agrees generally with the MC simulation over the gross range of the absorption changes. The model-prediction corresponding to the first layer 512a scattering not at the baseline presented some small non-monotonic patterns.

The following patterns of the diffuse reflectance corresponding to the CICA channel 700 and the CICB channel 704 when responding to the change of the reduced scattering of the first layer 512a may be appreciated. The MC simulation reveals that the diffuse reflectance of the CICA channel 700 may increase as the reduced scattering of the first layer 512a increases. Such increase is consistent with the response of the CIAD geometry to the increase of the dimensionless reduced scattering until reaching a saturation. At the same reduced scattering coefficient of the first layer 512a, the diffuse reflectance of the CICA channel 700 may decrease as the absorption of the first layer 512a increases. Whereas for the CICB channel 704, the diffuse reflectance may also reduce as the first layer 512a absorption increases. Yet the response of the CICB channel 704 to the increase of the reduced scattering of the first layer 512a may be opposite to that of the CICA channel 700. This decreasing pattern of the CICB channel 704 as the scattering of the first layer 512a increases may be related to the saturation of the diffuse reflectance of the CIAD geometry at a greater dimensionless reduced scattering (e.g., greater than 10) for the absorption level of the medium 504. Because of the saturation of the diffuse reflectance of the CIAD over a large radial dimension, any increase of the diffuse reflectance over a smaller radial dimension of the CIAD at the region corresponding to the CICA channel 700 may cause a reduction of the diffuse reflectance assessed over the annular area that is the difference between the greater radial dimension and the smaller radial dimension of the CICA channel 700. The difference is that of the CICB channel 704 which as is shown may respond to the change of the reduced scattering of the first layer 512a differentially in comparing to that of the CICA channel 700.

The model-predictions of the CICA channel 700 as a function of the first layer 512a scattering at the absorption of μₐ=0.1 and 1.0 mm⁻¹ agree generally with the MC simulation over the gross range of the scattering changes. Comparatively, the model-prediction of the CICB channel 704 as a function of the first layer 512a scattering at the absorption values assessed did reveal a patten of reduction as the scattering increased, however, the change was notably underestimated.

3.4.2. Experimental Responses of the CICA Channel 700 and the CICB Channel 704 to the Differential Contrasts of the Layers 512 with a 1.6 mm Thick First Layer 512a
3.4.2.1. a Medium 504 Consisting of a 1/16″ First Layer 512a with Second Layer 512b of Different Absorption

The medium 504 as measured consisted of a first layer 512a that was much more strongly absorbing in the sub-600 nm band than in the super-700 nm band, and a second layer 512b that was set at three different levels of absorption. The configurations of the medium 504 combined then would amount to the absorption-contrast to reduce at the sub-600 nm band and to increase over the super-700 nm band. The CICB channel 704 has shown much greater spectral change between the sub-600 nm band and the super-700 nm band than the CICA channel 700 does, for all three conditions of the medium 504. A major difference of the responses between the CICA channel 700 and the CICB channel 704 is the unleveled spectral profile of the CICA channel 700 considering that the dip around 550 nm must have been caused by the absorption of the first layer 512a of the medium 504. The unleveled pattern was attributed to the scattering dependency of the CICA channel 700 at its smaller size which would make it more sensitive to the scattering of the first layer 512a since the area-collected total diffuse is distant from the saturating zone. And the CICA channel 700 was also presented with a greater change of the spectral intensity over the super-700 nm band with respect to the sub-600 nm band. The model-prediction for the second layer 512b at the lowest absorption of 0.005 mm⁻¹ indicated a spectral dip at the sub-600 nm band, which was however unremarkable in the measurements. The model-prediction for the CICB channel 704 seemed to have significantly underestimated the spectral absorption at the sub-600 nm level.

### 3.5. Conclusions

We have demonstrated in this work a novel configuration of non-contact DRS implementing two channels presenting differing responsiveness to a two-layer medium 504 condition. The differential responsiveness, as would be resulted from two channels probing different depths, have been realized by combining a CICA geometry and a CICB geometry that are concentric with respect to the same POI 520. The CICA channel 700, due to its smaller area of light collection around the POI 520, probes and is more sensitive to the first layer 512a. And the CICB channel 704 collects light over an area that is significantly greater (i.e., approximately 1 order of magnitude greater) than that of the CICA channel 700 while also rejecting the light collected by the CICA channel 700. Such center-blocked configuration of the CICB channel 704 helps enhance the response to the contrast of absorption and scattering between the layers 512. MC simulations conducted with a 1 mm first layer 512a and experimental measurements specific to a 1.6 mm first layer 512a of complex absorption profile support the proposed principle of differential responsivity that may rendered by the complementary channel configurations. A preliminary model has shown promise, which however requires substantial improvement in accuracy.

### 4.1. Abstract

DRS relies upon the spectral responsivity of the photon remission to the medium's 504 property for application. Practically, it is imperative to assess the conditions at which the photon remission becomes less responsive or unresponsive to the change of an otherwise sensible property of the medium 504. Such limiting cases may be particularly relevant to SfR for minimally invasive sensing and CICA geometry for non-contact sensing. The steady-state photon remission of SfR in the absence of absorption, for example, was shown by MC to be insensitive to the scattering changes as the fiber-diameter scaled dimensionless reduced-scattering reaches

$${μ}_{s}^{′} {d}_{area} > 10.$$

Similar limiting patterns are observed with steady-state CICA known to be scalable over steady-state SfR. To gain insights on the conditions of saturation in CICA as projectable to SfR, we revise a model of the steady-state photon remission of CICA geometry described above, which predicts the onset of saturation as the dimensionless reduced scattering increases. The model-projected saturation-level and the corner-condition of the saturation, both being absorption-dependent, are examined against MC simulations. Experiments in non-contact CICA geometry were conducted. Diffuse reflectance was collected from two co-centric areas that differ by ˜10 times in diameter when responding to the same centered illumination. The limiting-pattern indicative model was applicable to the diffuse reflectance from aqueous samples of the scattering increased to near-saturation followed by absorption increasing. The scattering-saturation may be useful for simplifications such as implementing differential pathlength factor towards real-time assessment of absorption.

### 4.2. Introduction

DRS measures the remission of light diffusely propagated within (i.e., the reemitted light 518), thus interacting with a scattering medium 504 to inform the bulk properties of the medium 504. Nearly all DRS applications concern the spectral variation of absorption as the key information to resolve. For the cases of biological tissue, the spectral variation of the medium's 504 absorption properties may result from a static spectral difference or a dynamic temporal alteration. Large dynamic fluctuations of the absorption over time may occur endogenously due to hemoglobin oxygenation changes, and be induced exogenously by contrasting agents. Rating these changes of the medium 504 depends upon DRS to resolve the spectral attenuation that, however, is coupled between absorption and scattering. Therefore, understanding the condition of DRS when it becomes insensitive to the scattering changes is useful.

DRS involves an applicator-probe, whether by contact or non-contact means. The difference in the applicator-probe geometry distinguishes what is being measured, between spatially resolved photon remission and spatially integrated photon remission. If the applicator geometry permits collecting the diffuse photon remission over a spot off the POI 520, what is measured is the spatially resolved photon remission specific to a SDS or a multitude of SDSes. Such applicator geometry is commonly configured for contact measurement by light collection over one or multiple channels of fibers 804 that are separated from the channel for illumination. Such applicator geometry would not normally become unresponsive to scattering, except at some specialized SDSes that have shown scattering insensitivity useful to absorption characterization. The case of measuring spatially integrated diffuse photon remission has enabled applications including non-contact sensing of glucose in a CICA geometry. A geometry that may be much smaller in the physical dimension than the CICA geometry is SfR. Simple scaling of the steady-state photon remission between CICA and SfR suggests that the two geometries manifest similar patterns in terms of the dependence of the diffuse reflectance upon the change of the medium's 504 absorption and scattering properties.

The cross-coupling of scattering and absorption in DRS suggests that scattering saturation may be utilized to enhance the spectral specificity to absorption. Because DRS is a matter of the relative size of the geometry over the strength of scattering, the scattering saturation is alternatively a condition of the diffuse reflectance from a medium of fixed scattering versus further increase of the size of the light collection. These two conditions that may give the same dimensionless reduced scattering, however, differ due to the effect of absorption on the step-size of scattering. These two conditions leading to the same dimensionless reduced scattering, thus, may differ in the saturation behavior. For SfR, the saturation with respect to the dimensionless reduced scattering has been indicated via MC simulations. It has been shown that SfR saturates as the dimensionless reduced scattering

$${μ}_{s}^{′} {d}_{area} {exceeds}^{∼} 10 , where {μ}_{s}^{′}$$

is the reduced scattering coefficient and dₐᵣₑₐ is the diameter of the collection area. Similar values in terms of the level of saturation and

$${μ}_{s}^{′} {d}_{area}$$

signifying transition to saturation have also been observed with CICA. The development of the scattering insensitiveness versus the increase of the dimensionless reduced scattering, however, shows patterns incongruent between CICA and SfR. The saturation in CICA that is associated with increased size of collection over the same scattering reveals clear and fast transition from increasing to saturation. Comparatively, the saturation in SfR corresponding to the scattering increase over a fixed size of light collection is slow and not well-formed.

This work examines how the patterns of saturation of the total diffuse reflectance from CICA geometry may differ between two cases: (1) the scattering remains fixed while the size of light collection increases, and (2) the scattering increases while the size of light collection remains fixed. A model predicts a critical condition indicating the transition to scattering-saturation. The modeled saturation level and corner-parameter of the saturation transition are compared against the values deduced from MC simulations. Experiments acquired from CICA configurations differing in how the dimensionless reduced scattering is varied support the difference of the saturating patterns between the two cases.

### 4.3. Analytical Model

We model the total diffuse reflectance associated with an area of collection of a radius of rₐᵣₑₐ over a homogeneous semi-infinite medium 504 with the POI 520 at the origin, as is also illustrated in FIG. 5 and described above. The radially resolved diffuse reflectance may decrease as the radial distance increases. The total diffuse reflectance, or the diffuse reflectance collected over the area 524 centered on the POI 520, may increase as the diameter of the area 524 increases, until it saturates.

The resulted azimuthally uniform and radially resolved diffuse reflectance at a surface position having a radial distance of ρ from the POI 520 is R(ρ), where p∈[0,rₐᵣₑₐ]. The total diffuse photon remission over the entire circular area 524 having a radius rₐᵣₑₐ is then the integration of R(ρ) over the specific area 524 as the following:

$$I ( 0 ⇔ {r}_{area} ) = 2 π {\int }_{0}^{{r}_{area}} R ( {ρ}^{′} ) \cdot {ρ}^{′} \cdot d {ρ}^{′} \qquad ( 8 )$$

Apparently, the photon remission over the area of a radius of rₐᵣₑₐ is the difference between that over the entire surface 508 of the medium 504 and that falling outside the area of the radius of rₐᵣₑₐ. Equation (8) then becomes the following:

$$I ( 0 ⇔ {r}_{area} ) = 2 π [ {\int }_{0}^{∞} R ( {ρ}^{′} ) \cdot {ρ}^{′} \cdot d {ρ}^{′} - {\int }_{{r}_{area}}^{∞} R ( {ρ}^{′} ) \cdot {ρ}^{′} \cdot d {ρ}^{′} ] = {I}_{CIAD} ( 0 ⇔ ∞ ) - {I}_{CIAD} ( {r}_{area} ⇔ ∞ ) \qquad ( 9 )$$

$$where$$

$${I}_{CIAD} ( 0 ⇔ ∞ ) = 2 π {\int }_{0}^{∞} R ( {ρ}^{′} ) \cdot {ρ}^{′} \cdot d {ρ}^{′} \qquad ( 10 )$$

$${I}_{CIAD} ( {r}_{area} ⇔ ∞ ) = 2 π {\int }_{{r}_{area}}^{∞} R ( {ρ}^{′} ) \cdot {ρ}^{′} \cdot d {ρ}^{′} \qquad ( 11 )$$

are respectively the photon remission over the entire surface 508 of the medium 504 and that falling outside the area of collection with the radius of rₐᵣₑₐ. For later convenience, we take rₐᵣₑₐ=ζzₐ, where ζ is a dimensionless term, and zₐ is a reduced-scattering pathlength defined as zₐ=(μ′ₛ)⁻¹. Since I_{CIAD}(0⇔∞) and I_{CIAD}(rₐᵣₑₐ⇔∞) differ in only the lower limit of the integral, I_{CIAD}(0⇔∞) will be denoted as I_{CIAD}(rₐᵣₑₐ⇔∞) where δ=0, which will make it parallel with I_{CIAD}(ζzₐ⇔∞) that represents I_{CIAD}(rₐᵣₑₐ⇔∞). Equation (9) then appears as:

$$I ( δ {z}_{a} ⇔ ζ {z}_{a} ) = {I}_{CIAD} ( δ {z}_{a} ⇔ ∞ ) - {I}_{CIAD} ( ζ {z}_{a} ⇔ ∞ ) \qquad ( 12 )$$

The two parallel parts at the RHS of Equation (12) differ by only a dimensionless factor, either δ or ζ. If the scaling between the two parallel terms at the RHS of Equation (12) is represented with:

$$Δ ( δ , ζ ) = \frac{{I}_{CIAD} ( ζ {z}_{a} ⇔ ∞ )}{{I}_{CIAD} ( δ {z}_{a} ⇔ ∞ )} \qquad ( 13 )$$

we obtain an alternative and visually informative form of Equation (5) as the following:

$$I ( δ {z}_{a} ⇔ ζ {z}_{a} ) = {I}_{CIAD} ( δ {z}_{a} ⇔ ∞ ) [ 1 - Δ ( δ , ζ ) ] \qquad ( 14 )$$

we denote the ratio of absorption coefficient μₐ over reduced scattering coefficient of the medium as

$$γ = \frac{{μ}_{a}}{{μ}_{s}^{′}} \qquad ( 15 )$$

with which a few dimensionless terms relevant to modeling the diffuse reflectance are given as the following:

$${μ}_{eff} {z}_{a} = \sqrt{3 γ [ 1 + γ ]} \qquad ( 16 )$$

$$\frac{1}{D \cdot {μ}_{eff}} = \sqrt{\frac{3}{γ} ( 1 + γ )} \qquad ( 17 )$$

$${μ}_{eff} \cdot {d}_{area} = \sqrt{3 γ [ 1 + γ ]} \cdot ( {μ}_{s}^{′} \cdot {d}_{area} ) \qquad ( 18 )$$

where μ_{eff} is the effective attenuation coefficient [1/mm], and D is the diffusion coefficient [mm]. The

$${μ}_{s}^{′} \cdot {d}_{area}$$

in Equation (18) is the area of photon collection scaled over the reduced scattering pathlength.

It is apparent from Equation (14) that I(δzₐ⇔(zₐ) shall saturate at I_{CIAD}(δzₐ⇔∞) upon the zeroing of Δ(δ,ζ). The upper bound of Δ(δ, ζ) shall be 1 since physically I(δzₐ⇔ζzₐ) cannot be negative. However, the value of Δ(δ, ζ) may be mathematically unbound, as the result of the processes leading to the ratio term that may include approximation. The upper-bound of Δ(δ, ζ) could then indicate a critical condition or a corner-parameter of a limiting pattern—which should be the saturation. Furthermore, should Equation (14) be decomposed to linear combinations of sub-components, the saturation and the corner-condition of saturation then need to be treated as a weighted ensemble of those of the respective sub-components.

The first terms of the RHS of Equation (12) consists of four components:

$${I}_{CIAD} ( δ {z}_{a} ⇔ ∞ ) = {I}_{Ψ}^{master} ( δ {z}_{a} ⇔ ∞ ) + {I}_{Ψ}^{slave} ( δ {z}_{a} ⇔ ∞ ) + {I}_{Flux}^{master} ( δ {z}_{a} ⇔ ∞ ) + {I}_{Flux}^{slave} ( δ {z}_{a} ⇔ ∞ ) \qquad ( 19 )$$

where the four terms are the modeled contributions to the total photon remission over the specific area 524 of collection. The second term of the RHS of Equation (12) decomposes to the form of Equation (19), by simply replacing δ with ζ. Equation (12) thus can be formatted as the following:

$$I ( δ {z}_{a} ⇔ ζ {z}_{a} ) = {\sum }_{i = 1}^{4} {I}_{i} ( δ {z}_{a} ⇔ ζ {z}_{a} ) \qquad ( 20 )$$

where Iₖ(δzₐ⇔ζzₐ) is one of the four components of the RHS of Equation (19) subtracting its counterpart obtained by having δ replaced with ζ. All of the four parallel sections of Equation (20) can transform to the following pattern:

$${I}_{i} ( δ {z}_{a} ⇔ ζ {z}_{a} ) = {I}_{i}^{sat} \cdot [ 1 - {Δ}_{i} ] , i = [ 1 , 4 ] \qquad ( 21 )$$

The first terms of the RHS of the four equations of Equation (21) are respectively the following for i=[1, 4]:

$${I}_{i}^{sat} = {I}_{i}^{γ = 0} \cdot {ϕ}_{i} ( γ ) , i = [ 1 , 4 ] \qquad ( 22 )$$

which may be expanded as the following four expressions:

$${I}_{1}^{sat} = \frac{3}{8 \sqrt{2} π} 2 β \cdot ( \sqrt{\frac{3}{γ} ( 1 + γ )} ) \cdot exp ( - \sqrt{3 γ ( 1 + γ )} ) \cdot [ 1 - exp ( - \sqrt{3 γ ( 1 + γ )} \cdot 2 β ) ] , \qquad ( 23 )$$

$${I}_{2}^{sat} = S * \frac{3}{8 \sqrt{2} π} 2 β \cdot ( \sqrt{\frac{3}{γ} ( 1 + γ )} ) . [ 1 - exp ( - \sqrt{3 γ ( 1 + γ )} \cdot 2 β ) ] \qquad ( 24 )$$

$${I}_{3}^{sat} = \frac{3}{4 \sqrt{2} π} \cdot exp ( - \sqrt{3 γ ( 1 + γ )} ) \cdot [ 1 + exp ( - \sqrt{3 γ ( 1 + γ )} \cdot 2 β ) ] \qquad ( 25 )$$

$${I}_{4}^{sat} = S * \frac{3}{8 \sqrt{2} π} \cdot exp ( - \sqrt{3 γ ( 1 + γ )} ) \qquad ( 26 )$$

where S* is a scaling factor applied to the so-called slave or secondary model-source in the master-slave dual-source configuration. The four ratio-indicating terms in Equation (21) then are derived as the following for i=[1, 4]:

$${Δ}_{i} = \frac{{P}_{i}}{{μ}_{s}^{′} {d}_{a r e a}} exp [ - \frac{1}{2} \sqrt{3 γ ( 1 + γ )} \cdot ( {μ}_{s}^{′} \cdot {d}_{a r e a} ) ] \qquad ( 27 )$$

of which:

$${p}_{1} = 6.334 \frac{2 β \cdot \sqrt{3 γ [ 1 + γ ]}}{[ 1 - exp ( - β \cdot \sqrt{3 γ [ 1 + γ ]} ) ]} \cdot \frac{1}{exp ( - \sqrt{3 γ [ 1 + γ ]} )} \qquad ( 28 )$$

$${p}_{2} = 4.334 \frac{2 β \cdot \sqrt{3 γ [ 1 + γ ]}}{[ 1 - exp ( - β \cdot \sqrt{3 γ [ 1 + γ ]} ) ]} \qquad ( 29 )$$

$${p}_{3} = 6.334 \frac{1}{[ 1 + exp ( - β \cdot \sqrt{3 γ [ 1 + γ ]} ) ]} \cdot \frac{1}{exp ( - \sqrt{3 γ [ 1 + γ ]} )} \qquad ( 30 )$$

$${p}_{4} = 8.668 \frac{1}{[ 1 + exp ( - β \cdot \sqrt{3 γ [ 1 + γ ]} ) ]} \qquad ( 31 )$$

The numerical values in Equations (28)-(31) leading each fraction are the result of the refractive index difference across the medium-air interface. It is explicit that the LHS of Equation (21) shall reach the value limit of Equation (22) as

$${μ}_{s}^{′} {d}_{a r e a} ⇒ ∞$$

to make the ratio terms represented by Equation (31) vanish. As a result, the combined level of saturation is:

$${I}^{sat} = {lim}_{{μ}_{s}^{′} {d}_{a r e {a}^{⇒ ∞}}} I ( 0 ⇔ ζ {z}_{a} ) = {\sum }_{i = 1}^{4} {I}_{i}^{sat} \qquad ( 32 )$$

What Equation (20) represents are the contributions of the four modeled components to the total photon remission. And since each one represents a difference of the total diffuse collected over two areas with one being the sub-set of the other, the number as is expressed shall not be negative. This non-negativity, concerning Equation (21) that involves Equation (27), leads to the following condition at the limit of Δᵢ=1:

$${[ {μ}_{s}^{′} {d}_{a r e a} ]}_{i}^{crit} = {P}_{i} \cdot exp [ - \frac{1}{2} \sqrt{3 γ ( 1 + γ )} \cdot {[ {μ}_{s}^{′} {d}_{a r e a} ]}_{i} ] \qquad ( 33 )$$

The solution to Equation (33) will be a critical point which shall indicate a condition of importance—the transition to saturation. Equation (33) may be solved numerically to obtain the critical point of one of the four modeled components of the total photon remission. Since Equation (20) linearly combines four parts, the resulting critical point shall be the linear combination of the critical points obtained by Equation (33) that are weighted by the respective strength of the contribution of the individual part to the total diffuse reflectance. The combined critical value of the dimensionless reduced scattering is thus the following:

$${[ {μ}_{s}^{′} {d}_{a r e a} ]}_{}^{crit} = \frac{{{Σ}_{i = 1}^{4} [ {μ}_{s}^{′} {d}_{a r e a} ]}_{i}^{c r i t} \cdot {I}_{i}^{s a t}}{{Σ}_{i = 1}^{4} {I}_{i}^{s a t}} \qquad ( 34 )$$

Equation (34) predicts that there will be a value of

$${μ}_{s}^{′} {d}_{a r e a}$$

beyond which the total diffuse reflectance will saturate. Yet, Equation (33) involves the ratio of absorption over the reduced scattering parameter defined by Equation (15). This absorption dependent term will determine how distinct is the cross-over point. A rapidly crossing of the two sides will indicate a well-formed transition between two phases, whereas a slow crossing of the two sides will signal a poorly-formed transition between two phases. When

$${μ}_{s}^{′}$$

remains fixed while dₐᵣₑₐ increases, the LHS of Equation (33) increases monotonically and the RHS of Equation (33) decreases monotonically. That will make a sharp crossing of the two traces corresponding to both sides which shall indicate a well-defined transition. Comparatively, when

$${μ}_{s}^{′}$$

increases while dₐᵣₑₐ remain fixed, the LHS of Equation (33) increases monotonically as in the former case, however, the RHS of Equation (33) may not decrease monotonically due to the interplay between the square root term that is somewhat inversely proportional to

$${μ}_{s}^{′}$$

and the other term being proportional to

$${μ}_{s}^{′} .$$

This will cause a slower crossing of the two traces corresponding to both sides which shall indicate a loosely-defined transition.

### 4.4. Experimental Configuration

Two independent experimental setups, both in the CICA geometry, were used to assess the diffuse reflectance while varying the dimensionless reduced scattering in two different ways.

The details of one experimental setup conforming to the first case described above are shown in FIG. 9. The horizontal light-paths for 2-path fiber-coupled optics built with 1″ cage system may be reflected by one or more mirror 900 to vertical paths for projecting to or collecting from the leveled surface 508 of the medium 504. The illumination spot-size on the sample may be ˜0.5 mm in diameter. The size of light collection on the surface 508 of the medium 504 may be varied by adjusting the distance between the collection fiber and the focusing lens using a customized precision translation mechanism. By translating the collection fiber over 12 mm, the corresponding collection spot size on the medium 504 may be controlled to increase from a minimal size (e.g., 0.5 mm in diameter) to a maximum size (e.g., ˜25 mm in diameter). The conversion of the translation of the fiber-optical positioning at a resolution of 0.25 mm to the effective size of collection on the medium 504 may be based on the ray-tracing analysis of a similar setting. Diffuse reflectance may be acquired from two solid tissue phantoms. The two solid tissue phantoms may have nearly identical

$${μ}_{s}^{′} ( 1.04 {mm}^{- 1} and {mm}^{- 1} ) ,$$

and nearly 4 folds of difference in μₐ (0.0051 mm⁻¹ and 0.021 mm⁻¹).

Another experimental setup configured at an upright position shown in FIG. 8 conforms to the second case described above. The incident light 516 from the illumination source 220 may be coupled by the first fiber 804a to be projected normally onto the medium 504 via the 1″ cage optics consisting of the collimating lens 808 and the first relay lens 812a of 200 mm focal length. The reemitted light 518 may be acquired by two channels set at the opposite sides of the illuminating path and differing in the size of the area of collection. The CICA channel 700 may be aligned at an angle of approximately 22.5 degrees with respect to the optical axis of the illumination path. The CICB channel 704 may be aligned at an angle of approximately 30 degrees with respect to the optical axis of the illumination path. The CICA channel 700, which may comprise the one or more second lens (e.g., the second relay lens 812b (focal length=100 mm) and the first focusing lens 816a shown in FIG. 9), may collect the reemitted light 518 from the area 524 of, for example, 2.5 mm in diameter centered at the POI 520. The CICB channel 704, which may comprise the one or more third lens (e.g., the third relay lens 812c (focal length=100 mm) and the second focusing lens 816b shown in FIG. 9), may collect the reemitted light 518 from the center portion 128 of, for example, 25 mm in diameter, centered at the POI 520. The centers of the two channels may be aligned to overlap with the POI 520 at the surface level of the aqueous medium 504. The CICA channel 700 may be coupled via the second fiber 804b to the first compact spectrophotometer 820a. The CICB channel 704 may be coupled via the third fiber 804c to the second compact spectrophotometer 820b. Both spectrophotometers 820 may be controlled via their respective user interfaces. Exposure times of both spectrophotometers 820 may be the same at 300 ms. The spectral profiles over a 10 nm range centered at 620 nm from both spectrophotometers 820 may be averaged to obtain one value of the total diffuse reflectance of the channels, at a specific condition of the medium 504 of which the scattering was increased gradually.

The aqueous medium 504 of increasing scattering may be made by dissolving TiO₂ powders in 1000 mL water that is housed in a custom-made container having the second layer 512b of 1/16 inch thickness. The container with water may be placed on top of a magnetic stirrer to agitate the aqueous medium 504 using a 1.5″ stirrer at a speed of 180 RPM. The TiO₂ powder may be added to the solution to reach the following cumulative weights: 0.01 g to 0.1 g at an increment of 0.01 g, 0.1 g to 1 g at an increment of 0.1 g, 1 g to 10 g at an increment of 1 g, and 10 g to 100 g at an increment of 10 g. The reduced scattering coefficient of the aqueous medium 504 containing TiO₂ at 620 nm are estimated a

$${μ}_{s} ( λ ) = {μ}_{s}^{′} ( λ ) = 0.39 {λ}^{- 1.4} {mm}^{- 1} per mg / ml$$

of TiO₂, (wavelength in the unit of μm). At 620 nm, this gives

$${μ}_{s}^{′} = 0.7 \; 62 {mm}^{- 1} per mg / ml$$

of TiO₂. The baseline μₐ of the TiO2 solution at 620 nm may be set as 0.002 mm⁻¹.

### 4.5. Discussions

The pattern of the total diffuse reflectance associated with CICA in approaching saturation is shown to differ between the case of fixed reduced scattering with the size increasing (i.e., the first case described above) and the case of the scattering increasing with the size fixed (i.e., the second case described above). This difference may be appreciated by considering the different effects of absorption-increase to the total diffuse reflectance in the two cases of increasing the dimensionless reduced scattering to the same amount.

In the first case described above, the change of the dimensionless reduced scattering does not change the absorption over a pathlength over which a reduced scattering event happens. The increase of the light collection therefore gives a straight increase of the total light collected, until the size of collection reaches a scale significantly greater than the reduced scattering pathlength to collect almost all diffusely reemitted light 518. In this case, an increase of the absorption will have an across-the-board attenuation of the diffuse photon remission. Smaller value of the reduced scattering corresponds to relatively greater step size of reduced scattering. At smaller value of the reduced scattering, the size of the collection becomes relatively smaller compared to the reduced scattering pathlength to make the diffuse photon remission less sensitive to the change of the absorption. As the absorption increases, the stronger radial reduction of the diffuse photon remission may make the size of light collection reach sooner the condition of nearly completely collecting the diffuse photon remission, at only a lower value of the dimensionless reduced scattering. The saturation transition in this case is thus well-formed.

In the second case described above, the change of the dimensionless reduced scattering may change the absorption over a pathlength over which a reduced scattering event happens. The increase of the reduced scattering may decrease the step-size of the reduced scattering, over which the absorption attenuation reduces. This may cause relatively slower radial reduction of the diffuse photon remission over the area of collection. The increase of the dimensionless reduced scattering to drive the diffuse photon remission to a leveled value as would happen in the absence of absorption may be countered by a relative reduction of the absorption attenuation over the size of collection, which relaxes the leveling of the diffuse photon remission. The saturation transition in this case is thus loosely formed.

The dimensionless reduced scattering represents the size of the area of collection scaled over the reduced scattering pathlength. The insensitivity observed in SfR or CICA that occurs at

$${μ}_{s}^{′} {d}_{a r e a} > 10 ,$$

thus could impact the applications in one of two ways: (1) what is the limit of reduced scattering that cannot be sensed by a SfR/CICA of a fixed area? (2) what is the size of the area of detection that when surpassed leads to insensitivity to the reduced scattering? Such understanding will be instrumental to the selective applicability.

The change of the medium's 504 optical properties to be sensed may be either a static spectral difference or a dynamic temporal variation. Any diffuse reflectance measurements aiming to quantify medium 504 absorption properties usually deal with a large dynamic range of the absorption-associated signal variation. Large dynamic variations of the scattering over time may happen in cases such as tissue coagulation during energy-based treatment, as well as introduction of nano-scale agents for modulating tissue properties. Additionally, if the measurements will extend deeper into the short-wavelength spectrum, such as the ultraviolet band to take advantage of the Soret band of hemoglobin absorption, the tissue scattering will increase dramatically in comparison to that occurring in the near-infrared band. In such cases, whether the photon remission from the materials being assessed via diffusive optical probing will respond to the medium's scattering changes is not a trivial problem. And in the cases of the photon remission becoming unresponsive to one property of the medium 504, the variation of the photon remission thus will inform more specifically the changes of other properties of the medium 504. Such cases may be particularly desirable in more robustly decoupling the effects of multiple properties of the medium 504 on photon reemission.

### 4.6. Conclusions

This work develops an analytically augmented understanding of the patterns of saturation with respect to the increasing dimensionless reduced scattering, of the diffuse reflectance associated with CICA geometry. The approaching to the saturation is assessed in two cases over the same range of the dimensionless reduced scattering, one is by fixing the reduced scattering while changing the size of collection, and the other is by changing the reduced scattering while fixing the size of collection. The case of fixing the reduced scattering has shown a well-formed pattern of approaching the saturation. Such understanding of the saturation pattern may help application-specific configuration of DRS.

### 5.1. Abstract

Thin-film interference is commonly used for characterizing some physical properties of thin-film such as the thickness of it. Thin-film interference under broad-band light manifests spectrally varying periodicity, with the fringe becoming sparser towards longer wavelength, that informs the optical thickness of the thin-film. A non-scattering substrate of thin-film shall not alter the spectral periodicity of the thin-film interference due to no change to the pathlengths of interfering photons. A scattering substrate, however, may affect the fringe due to the contribution by photons of longer pathlengths than normal. And the proportion of the photons of longer pathlengths than normal may be affected by the diffusivity of the substrate. We observed that the spectrally varying periodicity of thin-film interference was affected by the substrate's diffusivity. Spectral thin-film interference was acquired from regular household food-wrap and oxygen-permeable film placed in good-contact with planar materials, by DRS in a CICA geometry over 550-850 nm. Spectral thin-film interference was compared among that acquired from film-attached Spectralon reflectance standards (40%, 60%, 80%, 99%) and film-covered solid tissue phantoms (near-identical reduced scattering with the absorption scaled 1:2:4). The variation of the spectral periodicity of thin-film interference can be associated with the scattering and absorption properties of the diffusive substrate. The effect of the diffusivity of the substrate on the spectral periodicity of thin-film interference may become a confounding issue for thin-film characterization but could provide information for probing thin-film covered materials towards applications including assessing surface and below-surface formation of MetMb.

### 5.2. Introduction

The interference of light transmitted through or reflected from a thin-film has been useful to characterizing a few radiative and geometric properties of the film, including refractive index, film thickness, and non-uniformity of the film thickness. When illuminated by a broad-band probing light, the thin-film interference reveals a wavelength-dependent periodicity, which may be exploited for assessing the substrate for applications such as thermal-radiative processes. If the substrate is a diffusive or turbid medium, the presence of the multiply scattered light in both the transmitted and reflected light field may complicate the interference fringe formation. The complication must reduce the visibility of the fringe patterns to degrade the sensitivity of fringe-based characterization. However, the complication might also modulate the fringe patterns that could help probe the substrate properties not amenable to probing otherwise.

This work reports observation that thin-film interference is affected by the scattering and absorption properties of the substrate of the thin-film.

### 5.3. Methods/Materials

The illumination source 220 was projected normally onto the medium 504. The reemitted light 518 was acquired by the CICA channel 700 and the CICB channel 704 set at the opposite sides of the illuminating path. The CICA channel 700 collected lights from an obliquely projected area of ˜2.5 mm in diameter centered at the POI 520. The CICB channel 704 collected lights from an area of ˜25 mm in diameter centered at the POI 520. The centers of the two channels were aligned to overlap with the POI 520 at the surface 508 of the medium 504. The data presented over 550-850 nm were associated with the CICA channel 700.

Spectral thin-film interference was acquired from regular household food-wrap and oxygen-permeable film placed in good-contact with planar materials. Spectral thin-film interference was compared among that acquired from film-attached Spectralon reflectance standards (i.e., 2%, 5%, 10%, 20%, 40%, 60%, 80%, 99%) and film-covered solid tissue phantoms having near-identical reduced scattering coefficient of 1.0 mm⁻¹ and the absorption coefficients of restively [0.005, 0.01, 0.021]mm⁻¹.

### 5.5. Discussion/Conclusion

The variation of the spectral periodicity of thin-film interference can be associated with the scattering and absorption properties of the diffusive substrate. The effect of the diffusivity of the substrate on the spectral periodicity of thin-film interference may become a confounding issue for thin-film characterization but could provide information for probing thin-film covered materials towards applications including assessing surface and below-surface formation of MetMb, or semi-conductor materials.

Referring back to FIG. 9, in some implementations, the incident light 516 from the illumination source 220 may travel through the first fiber 804a and the one or more first lens (e.g., the colimiting lens 808 and the first relay lens 812a shown in FIG. 9) to become the incident light 516, which is projected onto the surface 508 of the medium 504. A first portion of diffusely reflected light (i.e., the reemitting light 518 emitting from the medium 504 adjacent to the POI 520) may be shaped by the one or more second lens (e.g., the second relay lens 812b and the first focusing lens 816a shown in FIG. 9) and collected by the second fiber 804b. A second portion of diffusely reflected light (i.e., the reemitting light 518 emitting from the medium 504 farther away from the POI 520) may be reflected by the one or more mirror 900 to deflect the beam such that it may be shaped by the one or more third lens (e.g., the third relay lens 812c and the second focusing lens 816b shown in FIG. 9) and collected by the third fiber 804c. The center blocking module 600 may block the middle portion of the beam from being collected by the third fiber 804c. In this implementation, the incoming light path (i.e., the incident light 516) and the outgoing light path (i.e., the reemitting light 518 collected by the third fiber 804c) share the path below the one or more mirror 900.

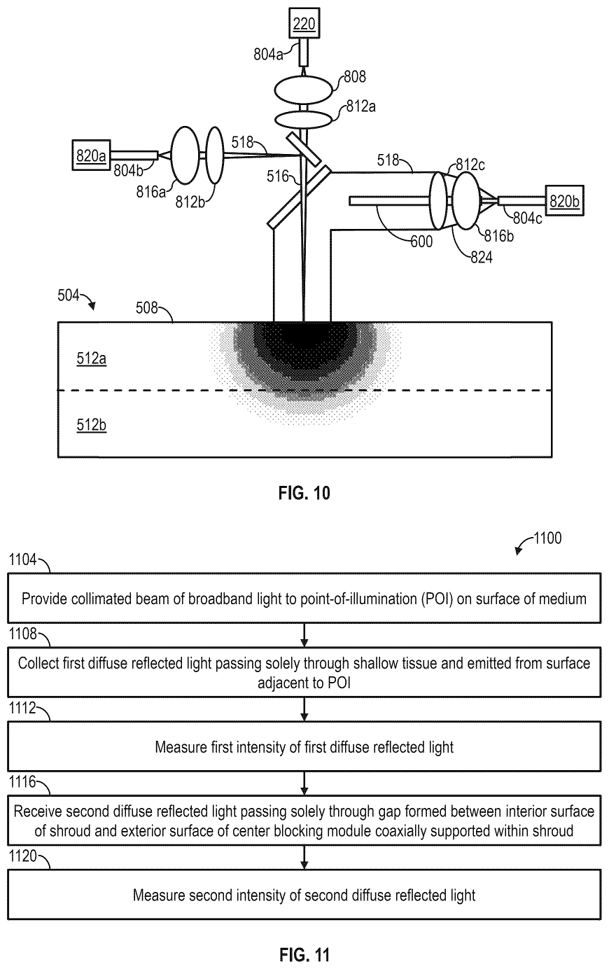

Referring now to FIG. 10, in some implementations, the incoming light path (i.e., the incident light 516) and both of the outgoing light paths (i.e., the reemitting light 518 collected by the second fiber 804b and the reemitting light 518 collected by the third fiber 804c) share the path below the one or more mirror 900. As shown in FIG. 10, in such implementations, the one or more mirror 900 may include a first mirror 900a configured to reflect the reemitting light 518 collected by the second fiber 804b and a second mirror 900b configured to reflect the reemitting light 518 collected by the third fiber 804c.

Referring now to FIG. 11, shown therein is a diagrammatic view of an exemplary implementation of a method 1100 of using the system 100 described herein. As shown in FIG. 11, the method 1100 generally comprises the steps of: providing, by an illumination source 220 of an absorption spectrophotometer 104, a collimated beam of broadband light (i.e., the incident light 516) to a POI 520 on a surface 508 of a medium 504, the medium 504 having a first layer 512a adjacent to the surface 508 and a second layer 512b beyond the first layer 512a (step 1104); collecting, by an optical element (i.e., the one or more second lens (e.g., the first focusing lens 816a and the second relay lens 812b) of a first spectrophotometer 820a of the absorption spectrophotometer 104, first diffuse reflected light (i.e., the reemitting light 518) passing solely through the first layer 512a and emitted from the surface 508 adjacent to the POI 520 (step 1108); measuring, by a first pixelated sensor 236 of the first spectrophotometer 820a, a first intensity of the first diffuse reflected light (step 1112); receiving, by a shroud 614 of a second spectrophotometer 820b of the absorption spectrophotometer 104, second diffuse reflected light (i.e., the reemitting light 518) passing solely through a gap formed between an interior surface of the shroud 614 and an exterior surface 616b of a center blocking module 600 coaxially supported within the shroud 614, the center blocking module 600 sized, shaped, and constructed of an opaque material so as to block the first diffuse reflected light passing solely through the first layer 512a and emitted from the surface 508 adjacent to the POI 520, the gap sized and shaped to pass the second diffuse reflected light passing through the second layer 512b at a predetermined range of distances from the POI 520 (step 1116); and measuring, by a second pixelated sensor 236 of the second spectrophotometer 820b, a second intensity of the second diffuse reflected light (step 1120). The data generated by the first spectrophotometer 820a and the second spectrophotometer 820b may be passed to the system device 108 and/or the user device 112, and/or analyzed by the processor(s) 200, 300 and/or 400 executing the software application 244 to determine one of more material property of the first layer 512a, and/or the second layer 512 as discussed herein.

### CONCLUSION

The foregoing description provides illustration and description, but is not intended to be exhaustive or to limit the inventive concepts to the precise form disclosed. Modifications and variations are possible in light of the above teachings or may be acquired from practice of the methodologies set forth in the present disclosure.

Even though particular combinations of features are recited in the claims and/or disclosed in the specification, these combinations are not intended to limit the disclosure. In fact, many of these features may be combined in ways not specifically recited in the claims and/or disclosed in the specification. Although each dependent claim listed below may directly depend on only one other claim, the disclosure includes each dependent claim in combination with every other claim in the claim set.

No element, act, or instruction used in the present application should be construed as critical or essential to the invention unless explicitly described as such outside of the preferred embodiment. Further, the phrase “based on” is intended to mean “based, at least in part, on” unless explicitly stated otherwise.

What is claimed is:

1. An absorption spectrophotometer in combination with a medium having a surface, a first layer adjacent to the surface, and a second layer beyond the first layer, comprising:
an illumination source operable to generate and provide a collimated beam of broadband light to a point-of-illumination (POI) on the surface of the medium;
a first spectrophotometer having an optical element operable to collect a diffuse reflected beam of light passing solely through the first layer of the medium and emitted from the surface of the medium adjacent to the POI, and a first pixelated sensor to measure intensity of the diffuse reflected beam of light; and
a second spectrophotometer comprising a shroud having an interior surface and a center blocking module having an exterior surface, the center blocking module coaxially supported within the shroud and forming a gap between the interior surface of the shroud and the exterior surface of the center blocking module, the center blocking module being sized, shaped, and constructed of an opaque material so as to block first diffuse reflected light passing solely through the first layer emitted from the surface of the medium adjacent to the POI, and the gap sized and shaped to pass second diffuse reflected light passing through the second layer and emitted from the surface of the medium at a predetermined range of distances from the POI, the second spectrophotometer also including a second pixelated sensor to measure intensity of the second diffuse reflected light emitted by the medium and passing through the gap between the interior surface of the shroud and the exterior surface of the center blocking module.

2. The absorption spectrophotometer in combination with the medium of claim 1, wherein the center blocking module has a first diameter and the shroud has a second diameter with the first diameter being less than the second diameter.

3. The absorption spectrophotometer in combination with the medium of claim 1, wherein the center blocking module includes a rod.

4. The absorption spectrophotometer in combination with the medium of claim 1, wherein the center blocking module includes a disc.

5. The absorption spectrophotometer in combination with the medium of claim 1, wherein the center blocking module has a first cross-sectional distance, and wherein the shroud has a second cross-sectional distance and wherein the first cross-sectional distance is in a range from 1/20 to ⅕ of the second cross-sectional distance.

6. The absorption spectrophotometer in combination with the medium of claim 5, wherein the second cross-sectional distance is an inner diameter of the shroud.

7. The absorption spectrophotometer in combination with the medium of claim 6, wherein the first cross-sectional distance is an outer diameter of the center blocking module.

8. The absorption spectrophotometer in combination with the medium of claim 1, wherein the medium has a first side and an opposed second side, and wherein the surface of the medium is on the first side of the medium, and wherein the illumination source, the first spectrophotometer and the second spectrophotometer are on the first side of the medium.

9. An absorption spectrophotometer, comprising:
a first spectrophotometer having an optical element operable to collect a diffuse reflected beam of light within a first field-of-view (FOV), and a first pixelated sensor to receive and measure intensity of the diffuse reflected beam of light; and
a second spectrophotometer comprising:
a shroud positioned to receive diffuse reflected light within a second FOV overlapping the first FOV and surrounding the first FOV, the shroud having an interior surface;
a center blocking module having an exterior surface, the center blocking module coaxially supported within the shroud and forming a gap between the interior surface of the shroud and the exterior surface of the center blocking module, the center blocking module being sized, shaped, and constructed of an opaque material so as to block first diffuse reflected light within a region of the second FOV overlapping the first FOV, the gap sized and shaped to pass second diffuse reflected light outside of the region overlapping the first FOV and within the second FOV;
a second pixelated sensor operable to measure intensity of the second diffuse reflected light passing through the gap between the interior surface of the shroud and the exterior surface of the center blocking module; and
an illumination source operable to generate and provide a collimated beam of broadband light to a point-of-illumination (POI) on a surface of a medium within the first FOV and the second FOV.

10. The absorption spectrophotometer of claim 9, wherein the center blocking module has a first diameter and the shroud has a second diameter with the first diameter being less than the second diameter.

11. The absorption spectrophotometer of claim 9, wherein the center blocking module includes a rod.

12. The absorption spectrophotometer of claim 9, wherein the center blocking module includes a disc.

13. The absorption spectrophotometer of claim 9, wherein the center blocking module has a first cross-sectional distance, and wherein the shroud has a second cross-sectional distance and wherein the first cross-sectional distance is in a range from 1/20 to ⅕ of the second cross-sectional distance.

14. The absorption spectrophotometer of claim 13, wherein the second cross-sectional distance is an inner diameter of the shroud.

15. The absorption spectrophotometer of claim 14, wherein the first cross-sectional distance is an outer diameter of the center blocking module.

16. The absorption spectrophotometer of claim 9, wherein the medium has a first side and an opposed second side, and wherein the surface of the medium is on the first side of the medium, and wherein the illumination source, the first spectrophotometer and the second spectrophotometer are on the first side of the medium.

17. A method, comprising:
providing, by an illumination source of an absorption spectrophotometer, a collimated beam of broadband light to a point-of-illumination (POI) on a surface of a medium, the medium having a first layer adjacent to the surface and a second layer beyond the first layer;
collecting, by an optical element of a first spectrophotometer of the absorption spectrophotometer, first diffuse reflected light passing solely through the first layer and emitted from the surface adjacent to the POI;
measuring, by a first pixelated sensor of the first spectrophotometer, a first intensity of the first diffuse reflected light;
receiving, by a shroud of a second spectrophotometer of the absorption spectrophotometer, second diffuse reflected light passing solely through a gap formed between an interior surface of the shroud and an exterior surface of a center blocking module coaxially supported within the shroud, the center blocking module sized, shaped, and constructed of an opaque material so as to block the first diffuse reflected light passing solely through the first layer and emitted from the surface adjacent to the POI, the gap sized and shaped to pass the second diffuse reflected light passing through the second layer at a predetermined range of distances from the POI; and
measuring, by a second pixelated sensor of the second spectrophotometer, a second intensity of the second diffuse reflected light.

18. The method of claim 17, wherein receiving the second diffuse reflected light is further defined as receiving, by the shroud of the second spectrophotometer of the absorption spectrophotometer, the second diffuse reflected light passing solely through the gap formed between the interior surface of the shroud and the exterior surface of the center blocking module coaxially supported within the shroud, the center blocking module sized, shaped, and constructed of the opaque material so as to block the first diffuse reflected light passing solely through the first layer and emitted from the surface adjacent to the POI, the gap sized and shaped to pass the second diffuse reflected light passing through the second layer at the predetermined range of distances from the POI, the center blocking module having a first diameter, the shroud having a second diameter, the first diameter less than the second diameter.

19. The method of claim 17, wherein receiving the second diffuse reflected light is further defined as receiving, by the shroud of the second spectrophotometer of the absorption spectrophotometer, the second diffuse reflected light passing solely through the gap formed between the interior surface of the shroud and the exterior surface of the center blocking module coaxially supported within the shroud, the center blocking module sized, shaped, and constructed of the opaque material so as to block the first diffuse reflected light passing solely through the first layer and emitted from the surface adjacent to the POI, the gap sized and shaped to pass the second diffuse reflected light passing through the second layer at the predetermined range of distances from the POI, the center blocking module having a first cross-sectional distance, the shroud having a second cross-sectional distance, the first cross-sectional distance in a range from 1/20 to ⅕ of the second cross-sectional distance.

20. The method of claim 19, wherein receiving the second diffuse reflected light is further defined as receiving, by the shroud of the second spectrophotometer of the absorption spectrophotometer, the second diffuse reflected light passing solely through the gap formed between the interior surface of the shroud and the exterior surface of the center blocking module coaxially supported within the shroud, the center blocking module sized, shaped, and constructed of the opaque material so as to block the first diffuse reflected light passing solely through the first layer and emitted from the surface adjacent to the POI, the gap sized and shaped to pass the second diffuse reflected light passing through the second layer at the predetermined range of distances from the POI, the center blocking module having the first cross-sectional distance, the shroud having the second cross-sectional distance, the first cross-sectional distance in the range from 1/20 to ⅕ of the second cross-sectional distance, the first cross-sectional distance being an outer diameter of the center blocking module, the second cross-sectional distance being an inner diameter of the shroud.
